SEQUENCE LISTING (6 sequences)

SEQ ID NO: 1 - protein, 8 aa, Synthetic construct
SIINFEKL

SEQ ID NO: 2 - protein, 8 aa, Synthetic construct
EIYQAGST

SEQ ID NO: 3 - protein, 15 aa, Synthetic construct
YTWFHAIHVSGTNGT

SEQ ID NO: 4 - protein, 9 aa, Synthetic construct
LALLLLDRL

SEQ ID NO: 5 - DNA, 20 bp, Synthetic construct
  source 1..20: mol_type = other DNA
tccatgacgttcctgatgct

SEQ ID NO: 6 - DNA, 24 bp, Synthetic construct
  source 1..24: mol_type = other DNA
tcgtcgttttgtcgttttgtcgtt

(19) United States
(12) Patent Application Publication
(10) Pub. No.: US 2025/0275922 A1
(43) Pub. Date: Sep. 4, 2025

(54) THERMOSTABLE PARTICLE-BASED COMPOSITIONS AND MANUFACTURING PROCESS

(71) Applicant: IOWA STATE UNIVERSITY RESEARCH FOUNDATION, INC., Ames, IA (US)

(72) Inventors: Balaji NARASIMHAN, Ames, IA (US); Kathleen A. ROSS, Ames, IA (US); Sean M. KELLY, Ames, IA (US); Michael J. WANNEMUEHLER, Ames, IA (US)

(73) Assignee: IOWA STATE UNIVERSITY RESEARCH FOUNDATION, INC., Ames, IA (US)

(21) Appl. No.: 19/068,558

(22) Filed: Mar. 3, 2025

Related U.S. Application Data
(60) Provisional application No. 63/560,441, filed on Mar. 1, 2024.

(51) Int. Cl. A61K 9/16; A61K 39/00; A61K 39/02; A61K 39/07; A61K 39/12; A61K 39/145; A61K 39/215; A61K 39/39

(52) U.S. Cl. CPC A61K 9/1682; A61K 39/0291; A61K 39/07; A61K 39/12; A61K 39/145; A61K 39/215; A61K 39/39; A61K 2039/55505; A61K 2039/55555; A61K 2039/55561

(57) ABSTRACT

Compositions and methods of using the same wherein the compositions include particles comprising one or more materials soluble in a solvent that is suitable for spray drying and one or more antigenic and/or non-antigenic payloads entrapped within the particles, wherein the composition is stable at room temperature for at least six months.

### RELATED APPLICATIONS

This application claims priority under 35 U.S.C. § 119 (e) to U.S. Provisional Patent Application No. 63/560,441 filed Mar. 1, 2024, which is incorporated herein by reference.

### SEQUENCE LISTING

The instant application contains a Sequence Listing which has been submitted electronically in XML format and is hereby incorporated by reference in its entirety. The XML file, created on Mar. 3, 2025, is named 509_019WO1_SL.xml, and is 6,164 bytes in size.

### BACKGROUND OF THE INVENTION

Particle-based vehicles (PBVs) represent a viable platform for the delivery of antigenic and non-antigenic payloads. In particular, polyanhydride PBVs are a promising system for the delivery of antigenic and non-antigenic payloads. These particles, comprised of copolymers of sebacic anhydride (SA), 1,6-bis(p-carboxyphenoxy)hexane (CPH), and 1,8-bis(p-carboxyphenoxy)-3,6-dioxaoctane (CPTEG), are biocompatible, demonstrating minimal injection-site reactivity when administered, and whose non-toxic dicarboxylic acid degradation products are safely excreted from the body. In addition, these materials have favorable amphiphilic properties that can preserve the stability and provide sustained release of encapsulated payloads via surface erosion kinetics. These formulations allow for enhanced shelf stability of encapsulated antigenic payloads for at least six months, even at elevated temperatures (e.g., 40° C.). Additionally, the inherent adjuvant properties of polyanhydrides promote robust cellular and humoral immune responses in vivo when used to deliver vaccine antigens. These properties have enabled the study of polyanhydride PBVs against multiple bacterial and viral pathogens.

PBVs are routinely synthesized via a solid-oil-oil flash nanoprecipitation (FNP) synthesis method. However, this methodology generally limits the particle batch size to laboratory scales of approximately 100 mg. In addition, FNP requires vast amounts of chilled non-solvents for particle precipitation, which is neither economical nor environmentally friendly. FNP synthesis also often results in aggregated particles as well as large variability in yields between particle batches dependent on the technician's skill. Finally, due to uneven distribution of large payload aggregates within the initial suspension prior to precipitation, the release kinetics and encapsulation efficiency can also vary greatly from batch to batch.

Despite the efficacy of many currently approved vaccines, most require storage at refrigeration temperature or below. These vaccines necessitate that the cold chain maintains consistent temperatures throughout the entire manufacturing, distribution, and long-term storage of national stockpiles. Additionally, refrigeration is very costly and often difficult to achieve in certain parts of the world, thus potentially reducing vaccine program success.

Accordingly, there is a need for PBVs that can be manufactured at scale with high batch-to-batch reproducibility. Additionally, there is a need for effective thermostable vaccines that can remain at room temperature without losing efficacy, and methods of making the same. The present disclosure satisfies these needs.

### SUMMARY OF THE INVENTION

The disclosure provides for particles comprising a material that is soluble in a solvent suitable for spray drying and one or more antigens or non-antigenic payloads entrapped within the particles, and methods of making the same.

In a first aspect, a method for manufacturing particle-based compositions comprises the steps of: i) reducing a lyophilized antigenic and/or a non-antigenic payload to an average size of about 10 nanometers to about 1500 nanometers in the presence of a first solvent to form a payload suspension; ii) mixing the payload suspension with a solution to form a mixture, wherein the solution comprises a material soluble in a second solvent, wherein the second solvent comprises a solvent suitable for spray drying; and iii) spray drying the mixture to form the particle-based composition, wherein the particles have an average size of about 0.15 μm to about 10 μm. In other aspects, the reducing step may comprise spray drying the antigenic or the non-antigenic payload to form a dry powder which can be suspended in the material solution.

In a second aspect, the reducing step comprises milling or grinding the lyophilized antigenic or the non-antigenic payload, and preferably, the milling comprises wet milling.

In a third aspect, the first solvent and the second solvent comprise one or more of an alcohol, a halogenated hydrocarbon, an aliphatic hydrocarbon, an aromatic hydrocarbon, a ketone, an ester, an ether, a nitrile, an amide, and acid, and a base.

In a fourth aspect, the first solvent and the second solvent comprise one or more of acetic acid, acetone, acetonitrile, benzene, 2-butanone, n-butanol, butyl acetate, carbon tetrachloride, chloroform, cyclohexane, 1,2-dichloroethane, dichloromethane, diethyl ether, di-isopropyl ether, dimethylformamide, dimethyl sulfoxide, dioxane, ethanol, ethyl acetate, heptane, hexane, isooctane, isopropanol, methanol, methyl ethyl ketone, methyl-t-butyl ether, pentane, n-propanol, tetrahydrofuran, toluene, trichloroethylene, water, and xylene.

In a fifth aspect, the first solvent and the second solvent comprise one or more of acetone, acetonitrile, chloroform, dichloromethane, ethanol, ethyl acetate, and isopropanol.

In a sixth aspect, the materials comprise one or more of a biodegradable material, a nonbiodegradable material, a chemically degradable material, a stimuli-responsive material, a water-soluble material, and a swellable material.

In a seventh aspect, the material comprises one or more of polyanhydrides, poly(lactic-co-glycolic acid) (PLGA), poly(lactic acid) (PLA), poly(glycolic acid) (PGA), polycaprolactone (PCL), poly(glycerol sebacate) (PGS), PEG-PCL, polyhydroxyalkanoates (PHA), polyethylene (PE), poly(methyl methacrylate) (PMMA), polytetrafluoroethylene (PTFE), silicone, polyurethane, poly(N-isopropylacrylamide) (PNIPAAm), poly(acrylic acid) (PAA), Pluronic, poly(diethyl amino ethyl methacrylate (PDEAEM), poly(ethylene glycol (PEG), poly(vinyl alcohol) (PVA), poly(2-hydroxyethyl methacrylate) (PHEMA), agarose, collagen, chitosan, alginate, hyaluronic acid, chondroitin sulfate, and gelatin.

In an eighth aspect, the antigenic payload comprises one or more of a protein, peptide, nucleic acid, and small molecule.

In a ninth aspect, the non-antigenic payload comprises one or more of an adjuvant, a protein, peptide, nucleic acid, small molecule, pharmaceutical drug, bacteria, virus, and whole cell lysate.

In a tenth aspect, a composition comprises particles comprising one or more materials soluble in a solvent suitable for spray drying; and one or more antigenic and/or non-antigenic payloads entrapped within the particles; wherein the one or more antigenic and/or non-antigenic payloads entrapped within the particles have an average size of about 10 nm to about 1500 nm, and the particles have an average size of about 0.15 μm to about 10 μm.

In an eleventh aspect, a composition comprises particles comprising one or more polymers soluble in a solvent suitable for spray drying; and one or more antigenic and/or non-antigenic payloads entrapped within the particles; wherein the particles have an average particle size of about 0.15 μm to about 10 μm.

In an twelfth aspect, a composition comprises particles comprising one or more polyanhydride polymers soluble in a solvent suitable for spray drying; and one or more antigenic and/or non-antigenic payloads entrapped within the particles; wherein the polyanhydride polymers have an average molecular weight of about 5,000 to 30,000 g/mol, the antigenic and/or the non-antigenic payload has an average size of about 10 nm to about 1500 nm, and the particles have an average particle size of about 0.15 μm to about 10 μm, wherein the particles are stable at room temperature for at least 6 months.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Characterization of PBV particle size and morphology during room temperature shelf storage. F1-V-loaded and PA-loaded polyanhydride PBVs were synthesized via FNP and stored at room temperature. Scanning electron micrographs demonstrate the particle size and morphology of room temperature stored PBVs were maintained similar to fresh PBVs.

FIG. 2A-D. Characterization of F1-V antigen released from extended room temperature stored PBVs. F1-V loaded polyanhydride PBVs synthesized via FNP were stored at room temperature for 33 months. The primary structure (A; SDS-PAGE), secondary structure (B; circular dichroism), tertiary structure (C; fluorescence spectroscopy), and antigenicity (D; ELISA) of released F1-V was found to be maintained when compared to a fresh F1-V control.

FIG. 3A-D. Characterization of PA antigen released from extended room temperature stored PBVs. PA loaded polyanhydride PBVs synthesized via FNP were stored at room temperature for 52 months. The primary structure (A; SDS-PAGE), secondary structure (B; circular dichroism), tertiary structure (C; fluorescence spectroscopy), and antigenicity (D; ELISA) of released PA was found to be maintained when compared to a fresh PA control.

FIG. 4A-B. Structural stability of payloads post-milling. (A) SDS-PAGE demonstrated the primary structure of F1-V, PA, SARS-COV-2 spike protein, RSV F protein, RSV G protein, and influenza A virus H5N1 HA trimer were preserved post-milling for approximately 18 h in ethanol. (B) Native PAGE demonstrated the native structure of F1-V, PA, and influenza A virus H5N1 HA trimer was also maintained compared to fresh protein controls.

FIG. 5A-C. Antigenicity of payloads post-milling. An ELISA demonstrated the antigenicity of milled (A) OVA, (B) BSA, and (C) influenza A virus H1N1 HA was preserved post-milling when compared to fresh protein controls.

FIG. 6. Size and morphology of spray dried payloads BSA and lysozyme. Example SEM micrographs demonstrate the discrete, spherical morphology of these payloads. The payload mean size+standard error of mean is shown below each micrograph (n=500 particles).

FIG. 7A-C. Size and morphology of PBV particles. Example SEM micrographs demonstrate the discrete, spherical morphology of 20:80 CPTEG:CPH PBVs regardless of payload. The (A) protein, (B) peptide (upper left: SIINFEKL (SEQ ID NO: 1); upper right: EIYQAGST (SEQ ID NO: 2); lower left: YTWFHAIHVSGTNGT (SEQ ID NO: 3); lower right: LALLLLDRL (SEQ ID NO: 4)), or (C) small molecule payload and mean size+standard error of mean is shown below each micrograph (n=500 particles).

FIG. 8A-C. Size distribution of PRET-synthesized PBVs. 20:80 CPTEG:CPH PBVs encapsulating (A) proteins, (B) peptides, or (C) small molecules were found to have similar size distributions regardless of encapsulated payload. Error bars represent the median particle size and inner quartiles of the analyzed particles (n=500 particles).

FIG. 9A-C. Release kinetics from PRET-synthesized PBVs. Spray dried 20:80 CPTEG:CPH particles were found to sustain the release of milled and encapsulated (A) proteins (filled circle: OVA; filled square: sheep IgG; filled triangle: H1N1 HA; open circle: BSA; open square: lysozyme; open triangle: H1N1 NP), (B) peptides (filled circle: SIINFEKL (SEQ ID NO: 1); filled square: LALLLLDRL (SEQ ID NO: 4); open circle: EIYQAGST (SEQ ID NO: 2); open square: YTWFHAIHVSGTNGT (SEQ ID NO: 3)), and (C) small molecules (filled circle: CpG 1668; filled square: doxycycline hyclate; open circle: CpG 2006; open square: cyclic-di-GMP).

FIG. 10. Structural stability of payloads following release from PBVs. SDS-PAGE demonstrated the primary structure of OVA, BSA, influenza A virus H1N1 HA, and influenza A virus H1N1 NP were preserved following release when compared to fresh protein controls.

FIG. 11. Antigenicity of payloads following release from PBVs. ELISA analysis of released antigens OVA and BSA demonstrated that antigenicity was preserved following release when compared to fresh protein controls.

FIG. 12. PRET synthesis of PBVs with various polymer formulations. SEM micrographs demonstrated PRET to be suitable for encapsulating 2% w/w OVA within multiple polymer formulations, resulting in discrete, spherical PBVs.

FIG. 13. Release kinetics of PBVs. PRET-synthesized PBVs sustain the release of encapsulated OVA protein regardless of polymer formulation; filled circle: 20:80 CPTEG:CPH; open circle: polySA; filled square: 10:90 CPTEG:SA; open square: 20:80 CPH:SA; filled triangle: 50:50 PLGA.

FIG. 14. SEM micrographs of spray dried PBVs with various polymer formulations encapsulating spray dried lysozyme nanoparticles (2% w/w), resulting in discrete, spherical PBVs. The mean particle size+standard error of mean is shown below each micrograph (n=100 particles).

FIG. 15. Co-encapsulation of multiple payloads within PBVs via PRET. PBV formulations of various polymer chemistries encapsulating 2% w/w OVA and 2% w/w CpG ODN 1668.

### DETAILED DESCRIPTION OF THE INVENTION

### Definitions.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001 or Singleton, et al., Dictionary of Microbiology and Molecular Biology, 2d ed., John Wiley and Sons, New York (1994), and Hale & Markham, The Harper Collins Dictionary of Biology. Harper Perennial, N.Y. (1991). General laboratory techniques (DNA extraction, RNA extraction, cloning, PCR amplification, cell culturing, etc.) are known in the art and described, for example, in Molecular Cloning: A Laboratory Manual, J. Sambrook et al., 4th edition, Cold Spring Harbor Laboratory Press, 2012.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five, or one to four, for example if the phenyl ring is disubstituted.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The term “about” can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, composition, or embodiment. The term about can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation or limitations which is not specifically disclosed herein.

As used herein, terms “subject” or “patient” are used interchangeably to refer to an animal (e.g., birds, reptiles, and mammals). In a specific embodiment, a subject is a bird. In another embodiment, a subject is a mammal including a non-primate (e.g., a camel, donkey, zebra, cow, pig, horse, goat, sheep, cat, dog, rat, and mouse) and a primate (e.g., a monkey, chimpanzee, and a human). In certain embodiments, a subject is a non-human animal. In another embodiment, a subject is a human.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

As used herein, “pharmaceutically acceptable” carrier or excipient includes any and all solvents, dispersion media, coatings, antibacterial and antifungal agents, isotonic and absorption/resorption delaying agents, and the like that are physiologically compatible. Preferably, the carrier is suitable for injection or infusion. Pharmaceutically acceptable carriers include sterile aqueous solutions or dispersions and sterile powders for the preparation of sterile injectable solutions or dispersion. The use of such media and agents for pharmaceutically active substances is known in the art. In addition to water, the carrier can be, for example, an isotonic buffered saline solution.

As used herein, “entrapped” or “encapsulated” refers to the incorporation or partial incorporation of an antigenic or non-antigenic payload, such as, but not limited to, an immunogenic protein and/or adjuvant into and/or onto the matrix of a biocompatible material. The properties of the material may be tailored for entrapment and delivery of the payload, such as, for example, a vaccine or therapeutic.

The terms “CpG oligodeoxynucleotide” and “CpG ODN” as used herein refer to a DNA molecule containing a dinucleotide of cytosine and guanine separated by a phosphate (also referred to herein as a “CpG” dinucleotide or “CpG”). The CpG ODN of the present disclosure contains at least one unmethylated CpG dinucleotide. That is, the cytosine in the CpG dinucleotide is not methylated (i.e., is not a 5-methylcytosine). The CpG ODN may have a partial or complete phosphorothioate (PS) backbone.

As used herein, the term “adjuvant” refers to a substance that modulates and/or generates an immune response, either directly or indirectly. Generally, an adjuvant is administered in conjunction with an antigen to result in an enhanced immune response to the antigen compared to the antigen alone. A variety of adjuvants are described herein.

As used herein, the term “soluble” refers to the ability of a substance (a solute) to dissolve and form a solution with a second substance (a solvent).

### Embodiments of the Invention

The present invention generally is directed to certain compositions, and in particular, certain immunogenic compositions, that may include particles comprising one or more materials (e.g., one or more polymers) that are soluble in a solvent suitable for spray drying, and one or more antigenic or non-antigenic payloads entrapped within the particles, wherein the one or more antigenic or non-antigenic payloads have an average size of about 10 nm to about 1500 nm and the particles have an average size of about 0.15 μm to about 10 μm.

In some embodiments, particles of the disclosure comprise materials that are soluble in a solvent that is suitable for spray drying such as, for example, a biodegradable material, a nonbiodegradable material, a chemically degradable material, a stimuli-responsive material, a water-soluble material, and a swellable material.

In some embodiments, the biodegradable material comprises one or more of collagen, chitosan, alginate, hyaluronic acid, chondroitin sulfate, and gelatin. In some embodiments, the biodegradable material comprises homopolymers or copolymers comprising one or more of polyanhydrides, polyesters, or another polymer soluble in a solvent suitable for spray drying. In some embodiments, the homopolymers or copolymers comprises CPTEG:CPH, CPTEG:SA, CPH:SA, polySA, poly(lactic acid) (PLA), poly(glycolic acid) (PGA), poly(lactic-co-glycolic acid) (PLGA), polycaprolactone (PCL), poly(glycerol sebacate) (PGS), PEG-PCL, and polyhydroxyalkanoates (PHA). (See Arpagaus et al., J. Pharm. Investig. 49, 405-426 (2019).)

In some embodiments, the stimuli-responsive material comprises one or more of poly(N-isopropylacrylamide) (PNIPAAm) and poly(acrylic acid) (PAA).

In some embodiments, the water swollen or swellable material comprises one or more of a pluronic comprising of hydrophilic poly(ethylene oxide) (PEO) and hydrophobic poly(propylene oxide) (PPO), poly(diethyl amino ethyl methacrylate) (PDEAEM), poly(ethylene glycol) (PEG), poly(vinyl alcohol) (PVA), poly(2-hydroxyethyl methacrylate) (PHEMA), and agarose.

In some embodiments, the non-biodegradable material comprises one or more of polyethylene (PE), poly(methyl methacrylate) (PMMA), polytetrafluoroethylene (PTFE), silicone, and polyurethane.

The prepared material can have average molecular weights of about 5,000 to about 55,000 g/mol, about 5,000 to about 45,000 g/mol, about 5,000 to about 40,000 g/mol, about 5,000 to about 35,000 g/mol, about 5,000 to about 30,000 g/mol, about 5,000 to about 25,000 g/mol, about 5,000 to about 20,000 g/mol, about 5,000 to about 15,000 g/mol, or about 5,000 to about 10,000 g/mol, or any amount or range therein. In other embodiments, the polymers or copolymers used to synthesize the particles have molecular weights of about 10,000 to about 30,000 g/mol, about 10,000 to about 27,500 g/mol, about 10,000 to about 25,000 g/mol, about 10,000 to about 22,500 g/mol, about 10,000 to about 20,000 g/mol, about 10,000 to about 17,500 g/mol, about 10,000 to about 15,000 g/mol, about 10,000 to about 12,500 g/mol, about 10,000 to about 12,000 g/mol, or any amount or range therein.

In various embodiments, the particles comprise polyanhydride homopolymers and/or copolymers of 1,ω-bis(p-carboxyphenoxy)alkanes and 1,ω-dicarboxylic alkanes such as 1,6-bis(p-carboxyphenoxy)hexane wherein every third carbon of the alkane moiety is optionally replaced with an oxygen to form one or more ethylene glycol moieties. Other polyanhydride copolymers preferably include poly(bis-(1,ω-carboxyphenoxy)(C₂-C₁₂)alkane-co-(C₅-C₂₀)bis-alkanoic acids). The substituents on the phenoxy moiety may be orientated ortho, meta, or para to each other, and are typically in a para relationship. The alkane moiety of the bis-(carboxyphenoxy) alkane may be a (C₂-C₁₂)alkane. In more specific embodiments, the alkane moiety may be a (C₄-C₈)alkane, and more specifically a (C₆)alkane. The alkanoic diacids used for the biodegradable polymers may be a (C₅-C₂₀)alkane bis-carboxylic acid. Specifically, the bis-carboxylic acid may be a (C₆-C₁₆)alkane bis-carboxylic acid, a (C₈-C₁₂)alkane bis-carboxylic acid, or more specifically a (C₁₀)alkane bis-carboxylic acid (i.e., sebacic acid). Optionally, the alkane and aryl moieties of the polyanhydride copolymers may be substituted in a manner that increases or decreases hydrophobicity of the particles.

A general formula for certain embodiments of the polyanhydride copolymers is:

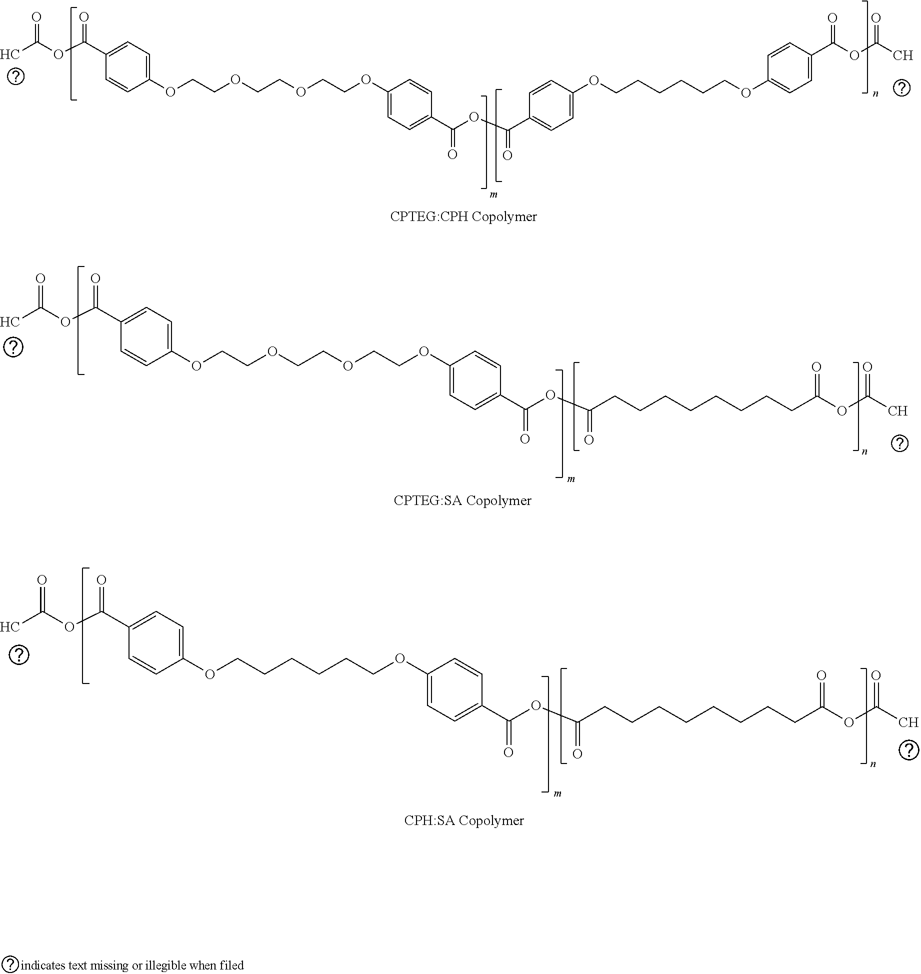

wherein m and n represent the number of repeating units of each monomer, for example, such that the polymers have molecular weights of about 4,000 to about 55,000 g/mol, or any amount or range therein. The variables “m” and “n” are not less than one and are typically greater than ten. In certain specific embodiments, polyanhydride homopolymers may also be used, in which case either m or n would be zero. In one specific embodiment, the polyanhydride is a copolymer of 1,8-bis(p-carboxyphenoxy)-3,6-dioxaoctane (CPTEG) and 1,6-bis(p-carboxyphenoxy)hexane (CPH) (abbreviated as CPTEG:CPH). In another embodiment, the polyanhydride is a copolymer of CPTEG and sebacic anhydride (SA) (abbreviated as CPTEG:SA). In another embodiment, the polyanhydride is a copolymer of CPH and SA (abbreviated as CPH:SA). The molecular weight range of the CPTEG:CPH copolymer is generally about 5,000 to about 30,000 g/mol, the molecular weight range of the CPTEG:SA copolymer is generally about 10,000 to about 30,000 g/mol, and the molecular weight range of the CPH:SA copolymer is generally about 10,000 to about 30,000 g/mol.

The polyanhydride copolymers may be synthesized through melt polycondensation from acetylated prepolymers using the techniques described by Kipper et al. (2002) Biomaterials 23, 4405-4412), Torres et al. (2006) J. Biomed. Mater. Res. A 76, 102-110), U.S. Pat. No. 7,858,093 (Kipper et al.), or U.S. Pat. No. 8,449,916 (Bellaire and Narasimhan), which describe examples of useful copolymers that can be used for the particles described herein, and which publications and patents are incorporated herein by reference. The polyanhydrides copolymers also may be prepared by microwave polymerization as described by Vogel et al. (2004) Macromol. Rapid Comm., 25, 330-333). Other techniques known to those of skill in the art also may be used to prepare the copolymers. The prepared polyanhydride copolymers can have molecular weights of about 5,000 to about 55,000 g/mol, about 5,000 to about 45,000 g/mol, about 5,000 to about 40,000 g/mol, about 5,000 to about 35,000 g/mol, about 5,000 to about 30,000 g/mol, about 5,000 to about 25,000 g/mol, about 5,000 to about 20,000 g/mol, about 5,000 to about 15,000 g/mol, or about 5,000 to about 10,000 g/mol, or any amount or range therein. In other embodiments, the polymers or copolymers used to fabricate the polymer particles have molecular weights of about 10,000 to about 30,000 g/mol, about 10,000 to about 27,500 g/mol, about 10,000 to about 25,000 g/mol, about 10,000 to about 22,500 g/mol, about 10,000 to about 20,000 g/mol, about 10,000 to about 17,500 g/mol, about 10,000 to about 15,000 g/mol, about 10,000 to about 12,500 g/mol, about 10,000 to about 12,000 g/mol, or any amount or range therein.

Preferably, the polyanhydride particles may degrade through surface-erosion due to the hydrolysis of anhydride linkages at the surface of the particle that may result in the controlled release of antigenic and/or non-antigenic payload to a subject. Surface-erodible biomaterials useful for the delivery of immunogens by the techniques disclosed herein are described by, for example, Narasimhan and Kipper, ((2004) Adv. Chem. Eng., 29, 169-218). Microstructural characterization of polyanhydride blends for controlled drug delivery are described by, for example, Mallapragada et al., Biomaterials for Drug Delivery and Tissue Engineering, Eds. (2001) Mater. Res. Soc. Symp. Proc. 662, NN4.2.1-4.2.5). Typically, particles of the invention are substantially spherical with an average size of about 0.15 μm to about 10 μm and a Polydispersity Index of about 0.1 to about 0.5.

Any suitable and effective ratio of monomers may be used in the synthesis of the particles. For example, certain embodiments include monomer ratios of about 1:1, about 1:1.5, about 1:2, about 1:3, about 1:4, about 1:5, and about 1:9. In some embodiments, the particles may comprise: the CPTEG:CPH copolymer in a molar ratio of about 20:80; the CPTEG:CPH copolymer in a molar ratio of about 50:50; the CPTEG:SA copolymer in a molar ratio of about 10:90; the PLGA copolymer in a molar ratio of about 50:50, the CPH:SA copolymer in a molar ratio of about 20:80, PolySA (homopolymer), PLA (homopolymer), PGA (homopolymer), 25:75 PLGA, 50:50 PLGA, or 75:25 PLGA. In some embodiments, a ratio of copolymers may be 1:10 to about 10:1, about 1:8 to about 8:1, about 1:7 to about 7:1, about 1:6 to about 6:1, about 1:5 to about 5:1, about 1:4 to about 4:1, about 1:3 to about 3:1, about 1:2 to about 2:1, or about 1:1.

In some embodiments, the material may comprise one or more of the following: lipids such phospholipids, triglycerides, cholesterol, and fatty acids; sugars and carbohydrates such as lactose, mannitol, trehalose, sucrose, and dextrose; proteins such as albumin, gelatin, and casein; salts and ionic compounds such as sodium chloride, calcium phosphate, and magnesium sulfate; silicon such as silicon dioxide; metals and metal oxides such as gold, iron oxide (magnetite), and titanium dioxide; ceramic such as hydroxyapatite and bioactive glass; amino acids such as leucine, glycine, and arginine, surfactants such as sodium dodecyl sulfate (SDS) and polysorbate; and waxes such as beeswax, carnauba wax, and paraffin wax.

In some embodiments, the average size of the antigenic or non-antigenic payload may be about 10 nm to about 1500 nm, about 50 nm to about 1250 nm, about 100 nm to about 1000 nm, about 200 nm to about 800 nm, or about 300 nm to about 600 nm, or any amount or range therein. In some embodiments, the average size of the antigenic or non-antigenic payload may be about 10 nm to about 1500, about 10 nm to about 1400 nm, about 10 nm to about 1300 nm, about 10 nm to about 1200 nm, about 10 nm to about 1100 nm, about 10 nm to about 1000 nm, about 10 nm to about 900 nm, about 10 nm to about 800 nm, about 10 nm to about 700 nm, about 10 nm to about 600 nm, about 10 nm to about 500 nm, about 10 nm to about 400 nm, about 10 nm to about 300 nm, about 10 nm to about 200 nm, about 10 nm to about 100 nm, about 10 nm to about 50 nm, or any amount or range therein. In other embodiments, the average size of the antigenic or non-antigenic payload may be about 50 nm to about 600 nm, about 75 nm to about 500 nm, about 100 nm to about 400 nm, any amount or range therein. In other embodiments, the average size of the antigenic or non-antigenic payload may be about 150 nm to about 1200 nm or any amount or range therein. In other embodiments, the average particle size of the antigenic or non-antigenic payload may be about 500 nm to about 1500 nm, about 600 nm to about 1200 nm, or any amount or range therein. In some embodiments, the average size of the antigenic or non-antigenic payload may be about 950 nm to about 1800 nm.

As noted, the particles may be constructed of any material that is soluble in a solvent that is suitable for spray drying. For example, such solvents may include, but are not limited to one or more of an alcohol, a halogenated hydrocarbon, an aliphatic hydrocarbon, an aromatic hydrocarbon, a ketone, an ester, an ether, a nitrile, an amide, an acid, and a base.

In some embodiments, the solvent suitable for spray drying comprises one or more of acetic acid, acetone, acetonitrile, benzene, 2-butanone, n-butanol, butyl acetate, carbon tetrachloride, chloroform, cyclohexane, 1,2-dichloroethane, dichloromethane, diethyl ether, di-isopropyl ether, dimethylformamide, dimethyl sulfoxide, dioxane, ethanol, ethyl acetate, heptane, hexane, isooctane, isopropanol, methanol, methyl ethyl ketone, methyl-t-butyl ether, pentane, n-propanol, tetrahydrofuran, toluene, trichloroethylene, water, and xylene.

In some embodiments, the solvent suitable for spray drying comprises one or more of acetone, acetonitrile, chloroform, dichloromethane, ethanol, ethyl acetate, and isopropanol.

In some embodiments, the materials, such as but not limited to biocompatible polymers, may be used to entrap one or more antigens, and, optionally, one or more adjuvants for delivery to a subject. As used herein, the term “antigen” refers to a molecule with one or more epitopes that stimulate a host's immune system to make a secretory, humoral and/or cellular antigen-specific response against one or said antigen. The term is also used interchangeably with “immunogen”. By way of example, embodiments may include one or more specific antigens that may be a complete protein, portions of a protein, a peptide, fusion proteins, glycosylated proteins, and combinations thereof. In some embodiments, the antigenic payload comprises a protein, peptide, nucleic acid, or small molecule.

Exemplary antigens can include any eukaryotic antigen or prokaryotic antigen such, for example, the antigens described in U.S. Pat. No. 8,927,024 to Bellaire et al.; U.S. Pat. No. 10,874,737 to Narasimhan et al.; U.S. Pat. No. 11,484,599 to Hubbell et al.; and U.S. Patent Publication Number 2015/0216888 to Bellaire et al. For example, antigens for use with the particles may include antigenic proteins from one or more serotypes of Influenza Virus such as Influenza A virus, Influenza B virus, Influenza C virus, and/or Influenza D virus. Other particle embodiments may include a mixture of antigens from two or more Influenza serotypes (e.g. Influenza A Virus and Influenza B virus).

Certain embodiments of particles may include antigens from only a single Influenza serotype (e.g. only Influenza A Virus). Alternatively, particles may include the immunogenic proteins hemagglutinin (H1, H2, H3, H4, H5, H6 H7, H8, H9, H10, H12, H13, H14, H15, H16, H17, and H18) and neuraminidase (N1, N2, N3, N4, N5, N6, N7, N8, N9, N10, and N11) from certain Influenza A virus subtypes.

Exemplary immunogens from influenza A, influenza B, Influenza C, and influenza D virus include also Hemagglutinin (HA) (subtypes H1-H18, HA Yamagata, HA Victoria, H1N1 strains A/Albany/12/1951, A/Beijing/22808/2009, A/Beijing/262/1995, A/Brevig Mission/1/1918, A/Brisbane/59/2007, A/California/04/2009, A/California/06/2009, A/California/07/2009, A/Chile/1/1983, A/England/195/2009, A/England/42/1972, A/New Caledonia/20/1999, A/New York/06/2009, A/New York/1/1918, A/New York/18/2009, A/New Jersey/8/1976, A/Ohio/07/2009, A/Ohio/UR06-0091/2007, A/Puerto Rico/8/1934, A/Puerto Rico/8/34/Mount Sinai, A/Solomon Islands/3/2006, A/swine/Belgium/1/1998, A/Swine/Wisconsin/136/1997, A/Taiwan/01/1986, A/Texas/05/2009, A/Texas/36/1991, A/USSR/90/1977, A/USSR/92/1977, A/WSN/1933, A/Wilson-Smith/33, A/Tientsin/78/77, A/Singapore/6/86, A/Memphis/39/83, A/Malaysia/54, A/Iowa/43, A/Hong Kong/117/77, A/Fort Monmouth/1/47, A/Baylor/4052/81, A/Albany/4835/48; H1N2 strain A/swine/Guangxi/13/2006, A/Singapore/1/1957; H1N3 strain A/duck/NZL/160/1976; H2N2 Strain A/Ann Arbor/6/1960, A/Canada/720/2005, A/Guiyang/1/1957, A/Japan/305/1957; H3N2 strain A/Aichi/2/1968, A/Babol/36/2005, A/Brisbane/10/2007, A/California/7/2004, A/Chiang Rai/277/2011, A/Christchurch/4/1985, A/Fujian/411/2002, A/Guangdong-Luohu/1256/2009, A/HongKong/1/1968, A/Hong Kong/CUHK31987/2011, A/Indiana/07/2012, A/Memphis/1/68, A/Moscow/10/1999, A/New York/55/2004, A/Perth/16/2009, A/reassortant/IVR-155, A/Sydney/5/1997, A/Texas/50/2012, A/Victoria/208/2009, A/Victoria/210/2009, A/Victoria/3/1975, A/Victoria/361/2011, A/Wisconsin/15/2009, A/Wisconsin/67/X-161/2005, A/Wyoming/03/2003, A/X-31; H3N8 strains A/canine/New York/145353/2008, A/equine/Gansu/7/2008; H4N2 strain A/duck/Hunan/8-19/2009; H4N4 A/mallardduck/Alberta/299/1977, H4N6 A/mallard/Ohio/657/2002, A/Swine/Ontario/01911-1/99; H4N8 A/chicken/Alabama/1/1975; H5N1 strain, A/HongKong/156/97, A/chicken/Shanxi/2/06, A/silky chicken/Hong Kong/SF189/01, A/chicken/Henan/16/04 (H5N1), A/Anhui/1/2005, A/bar-headedgoose/Qinghai/14/2008, A/bar-headedgoose/Qinghai/1A/2005, A/barnswallow/Hong Kong/D10-1161/2010, A/Cambodia/R0405050/2007, A/Cambodia/S1211394/2008, A/chicken/Egypt/2253-1/2006, A/chicken/India/NIV33487/2006, A/chicken/Jilin/9/2004, A/chicken/VietNam/NCVD-016/2008, A/chicken/Yamaguchi/7/2004, A/Common magpie/HongKong/2256/2006, A/commonmagpie/Hong Kong/5052/2007, A/Duck/HongKong/p46/97, A/duck/Hunan/795/2002, A/duck/Laos/3295/2006, A/Egypt/2321-NAMRU3/2007, A/Egypt/3300-NAM RU3/2008, A/Egypt/N05056/2009, A/goose/Guangdong/1/96, A/goose/Guiyang/337/2006, A/Hongkong/213/03, A/HongKong/483/97, A/Hubei/1/2010, A/Hubei/2011, A/hubei/2011-CDC, A/Indonesia/5/2005, A/Japanesewhite-eye/HongKong/1038/2006, A/Thailand/1 (KAN-1)/2004, A/turkey/Turkey/1/2005, A/Vietnam/UT31413II/2008, A/whooper swan/Mongolia/244/2005, A/Xinjiang/1/2006; H5N2 strain A/American green-winged teal/California/HKWF609/07, A/ostrich/South Africa/AI1091/2006; H5N3 strain A/duck/Hokkaido/167/2007; H5N8 strain A/breeder duck/Korea/Gochang1/2014, A/broilerduck/Korea/Buan2/2014, A/duck/Jiangsu/k1203/2010, A/duck/NY/191255-59/2002, A/duck/Zhejiang/6D18/2013, A/duck/Zhejiang/W24/2013, A/turkey/Ireland/1378/1983; H5N9 strain A/chicken/Italy/22A/1998; H6N1 strain A/northern shoveler/California/HKWF115/2007; H6 N4 strain A/chickenHongKong/17/77; H6N5 strain A/shearwater/Australia/1/1973; H6N6 strain A/duck/Eastern China/11/2009; H6N8 strain A/mallard/Ohio/217/1998; H7N1 strain A/turkey/Italy/4602/99; H7N2 strain A/ruddy turnstone/New Jersey/563/2006, H7N3 strain A/chicken/SK/H R-00011/2007, A/turkey/Italy/214845/2002; H7N7 strain A/chicken/Netherlands/1/03, A/equine/Kentucky/1a/1975, A/Netherlands/219/2003; H7N8 strain A/mallard/Netherlands/33/2006; H7N9 strain A/Anhui/1/2013, A/Anhui/PA-1/2013, A/chicken/Zhejiang/DTID-ZJU01/2013, A/Hangzhou/1/2013, A/Hangzhou/3/2013, A/Huzhou/10/2013, A/Pigeon/Shanghai/S1069/2013, A/Shanghai/1/2013, A/Shanghai/4664T/2013, A/Shanghai/Patient3/2013, A/Zhejiang/1/2013, A/Zhejiang/DTID-ZJU10/2013; H8N4 strain A/pintail duck/Alberta/114/1979; H9N2 strain A/brambling/Beijing/16/2012, A/Chicken/Hong Kong/G9/1997, A/duck/Hong Kong/448/78, A/Guinea fowl/Hong Kong/WF10/99, A/Hong Kong/1073/99, A/Hong Kong/2108/2003, A/Hong Kong/3239/2008, A/Hong Kong/35820/2009; H9N5 strain A/shorebird/DE/261/2003; H9N8 strain A/chicken/Korea/164/04; H10N3 strain A/duck/Hong Kong/786/1979, A/duck/Hunan/S11205/2012, A/mallard/Minnesota/Sg-00194/2007; H10N4 strain A/mink/Sweden/3900/1984; H10N7 strain A/blue-winged teal/Louisiana/Sg-00073/2007; H10N8 strain A/duck/Guangdong/E1/2012, A/Jiangxi-Donghu/346/2013; H10N9 strain A/duck/HongKong/562/1979, A/duck/Hong Kong/562/1979; H11N2 strain A/duck/Yangzhou/906/2002, A/thick-billed murre/Newfoundland/031/2007; H11N6 strain A/duck/England/1/1956; H11N9 strain A/mallard/Alberta/294/1977; H12N1 strain A/mallard duck/Alberta/342/1983; H12N3 strain A/bar headed goose/Mongolia/143/2005; H12N5 strain A/green-winged teal/ALB/199/1991; H13N6 strain A/black-headed gull/Sweden/1/1999; H13N8 A/black-headed gull/Netherlands/1/00; H14N5 strain A/Mallard/Astrakhan (Gurjev)/263/1982; Australia/1756/1983; H15N2 strain A/Australian shelduck/Western H15N2 strain A/duck/AUS/341/1983; H16N3 strain A/black-headed gull/Sweden/5/99; H17N10 strain A/little yellow-shouldered bat/Guatemala/164/2009; H18N11 strain A/flat-faced bat/Peru/033/2010), Influenza B Virus strain B/Brisbane/3/2007, B/Brisbane/60/2008, B/Florida/07/2004, B/Florida/4/2006, B/Hong Kong/05/1972, B/Malaysia/2506/2004, B/Massachusetts/03/2010, B/Ohio/01/2005, B/PHUKET/3073/2013, B/Utah/02/2012, B/Victoria/02/1987, B/Victoria/504/2000, B/Wisconsin/01/2012, B/Yamagata/16/1988; Neuraminidase (NA) (subtypes N1-N11, NA Yamagata, NA Victoria), Nucleocapsid Protein (NP), Matrix Protein 1 (M1), Matrix Protein 2 (M2), Polymerase Basic Protein 1 (PB1), Polymerase Basic Protein 2 (PB2), Polymerase Acidic Protein (PA), Nonstructural Proteins 1 (NS1), Nonstructural Proteins 2/Nuclear Export Protein (NS2/NEP), Polymerase Basic Protein 1 Segment Second Proteins (PB1-F2), Influenza B Virus Membrane Protein (BM2), Influenza B Virus Membrane Protein (NB), Influenza A Virus Segment 2 Alternative Splicing Protein (M42), Influenza A Virus Segment 1 Alternative Splicing Protein (PB2-S1), Influenza A Virus Segment 2 Alternative Initiation Protein (N40)), Influenza A Virus Segment 3Ribosomal Shift Protein (PA-X), Influenza A Virus Segment 3 Alternative Initiation Protein (PA-N182), Influenza A Virus Segment 3 Alternative Initiation Protein (PA-N155), Influenza C/D Virus Polymerase Complex Protein (P3), Influenza C/D Virus Surface Glycoproteins: Hemagglutinin, Esterase, and Fusion activities (HEF), Influenza C/D Virus Matrix Protein (CM1), or Influenza C/D Virus surface glycoprotein CM2.

In some embodiments, influenza A virus immunogenic proteins include HA (subtypes H1, H2, H3, H4, H5, H6, H7, H8, H9, H10, H11, H12, H13, H14, H15, H16, H17, H18), NA (subtypes N1, N2, N3, N4, N5, N6, N7, N8, N9, N10, N11), NP, M1, M2, PB1, PB2, NS1, NS2/NEP, PA, PB1-F2, M42, PB2-S1, N40, PA-X, PA-N182, and PA-N155; influenza B virus immunogens include HA Yamagata, HA Victoria, NA Yamagata, NA Victoria, NP, M1, M2, PB1, PB2, NS1, NS2/NEP, PA, and NB; influenza C virus immunogens include HEF, NP, M1, M2, PB1, PB2, NS1, NS2/NEP, and P3; influenza D virus immunogens include HEF, NP, M1, M2, PB1, PB2, NS1, NS2/NEP, and P3.

Other non-limiting examples of viral antigens include, but are not limited to, e.g., retroviral antigens such as retroviral antigens from the human immunodeficiency virus (HIV) antigens such as gene products of the gag, pol, and env genes, the Nef protein, reverse transcriptase, and other HIV components; hepatitis viral antigens such as the S, M, and L proteins of hepatitis B virus, the pre-S antigen of hepatitis B virus, and other hepatitis, e.g., hepatitis A, B, and C, viral components such as hepatitis C viral RNA; influenza viral antigens such as hemagglutinin and neuraminidase and other influenza viral components; measles viral antigens such as the measles virus fusion protein and other measles virus components; mumps virus antigens; rubella viral antigens such as proteins E1 and E2 and other rubella virus components; rotaviral antigens such as VP7sc and other rotaviral components; cytomegaloviral antigens such as envelope glycoprotein B and other cytomegaloviral antigen components; respiratory syncytial viral antigens such as the RSV fusion (F) protein, glycoprotein (G), the M2 protein and other respiratory syncytial viral antigen components; herpes simplex viral antigens such as immediate early proteins, glycoprotein D, and other herpes simplex viral antigen components; varicella zoster viral antigens such as gpI, gpII, and other varicella zoster viral antigen components; Japanese encephalitis viral antigens such as proteins E, M-E, M-E-NS1, NS1, NS1-NS2A, 80% E, and other Japanese encephalitis viral antigen components; rabies viral antigens such as rabies glycoprotein, rabies nucleoprotein and other rabies viral antigen components; picornavirus antigens, such as enteroviruses, rhinoviruses, heparnavirus, cardioviruses and aphthoviruses; filoviruses such as Ebola and Marburg virus; hantavirus pulmonary syndrome virus; viral antigens derived from a pestivirus, such as bovine viral diarrhea (BVDV), classical swine fever (CSFV) or border disease (BDV); or antigens derived from a coronavirus, Severe acute respiratory syndrome (SARS-COV), Middle East respiratory syndrome (MERS-COV), human respiratory coronavirus, avian infectious bronchitis (IBV), mouse hepatitis virus (MHV), SARS-COV-2, and porcine transmissible gastroenteritis virus (TGEV). See, Vaccines, 6th Edition (Plotkin, Orenstein and Offit ed. 2012); Medical Microbiology 8th Edition (Murray, et al. ed. 2015) Fundamental Virology, 4th Edition, (Knipe, D. M., et al. eds. 2001) for additional examples of viral antigens. Other viral antigens include antigens from one or more of orthomyxoviridae, paramyxoviridae, coronaviridae, filoviridae, flaviviridae, hantaviridiae, and pneumoviridae viruses.

Non-limiting examples of bacterial antigens include, but are not limited to, e.g., bacterial antigens such as pertussis toxin, filamentous hemagglutinin, pertactin, FIM2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components; streptococcal bacterial antigens such as M proteins and other streptococcal bacterial antigen components; Staphylococcal antigens, such as Staphylococcus aureus antigens, gram-negative bacilli bacterial antigens such as lipopolysaccharides and other gram-negative bacterial antigen components, Mycobacterium tuberculosis bacterial antigens such as mycolic acid, heat shock protein 65 (HSP65), the 30 kDa major secreted protein, antigen 85A and other antigen components; Helicobacter pylori bacterial antigen components; mycobacterial pneumococcal bacterial antigens such as pneumolysin, pneumococcal capsular polysaccharides and other pneumococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens. Partial or whole pathogens may also be: Haemophilus influenza; Plasmodium falciparum; Neisseria meningitidis; Streptococcus pneumoniae; Neisseria gonorrhoeae; salmonella serotype typhi; shigella; Vibrio cholerae; Dengue Fever; Encephalitides; Japanese Encephalitis; Lyme disease; Yersinia pestis; west Nile virus; yellow fever; Zika virus; tularemia; hepatitis (viral; bacterial); RSV (respiratory syncytial virus); HPIV 1 and HPIV 3; adenovirus; small pox; allergies and cancers.

Non-limiting examples of fungal antigens include, but are not limited to, e.g., candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Examples of protozoal and other parasitic antigens include, but are not limited to, e.g., Plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; Leishmania major and other leishmaniae antigens such as gp63, lipophosphoglycan and its associated protein and other leishmanial antigen components; and Trypanosoma cruzi antigens such as the 75-77 kDa antigen, the 56 kDa antigen and other trypanosomal antigen components; malaria antigens such as Plasmodium Glutamate dehydrogenase, histidine rich protein II, lactate dehydrogenase, and aldolase.

The antigen may also be one or more of viruses (inactivated, attenuated, and modified live), bacteria, parasites, nucleotides, polynucleotides, peptides, polypeptides, recombinant proteins, synthetic peptides, protein extract, cells (including tumor cells), tissues, polysaccharides, carbohydrates, fatty acids, teichoic acid, peptidoglycans, lipids, or glycolipids, individually or in any combination thereof. The antigen may be wild-type or mutated.

The antigens used in methods and compositions described herein also include immunogenic fragments of nucleotides, polynucleotides, peptides, and polypeptides that can be isolated from the organisms referred to herein.

In other embodiments, an antigen comprises one of more Yersinia pestis fusion protein F1-V, Bacillus anthracis PA antigen, hen-egg ovalbumin (OVA), hen-egg lysozyme, hemagglutinin H5 trimer, influenza virus hemagglutinin (HA), influenza virus nucleoprotein (NP), RSV pre-or post-fusion F protein, RSV G protein, SARS-COV-2 S protein, SARS-COV-2 N protein, SARS-CoV-2 M protein, SARS-COV-2 E protein. SARS-COV-2 variants include Alpha (B.1.1.7, Q.1-Q.8), Beta (B.1.351, B.1.351.2, B.1.351.3), Delta (B.1.617.2), Epsilon (B.1.427, B.1.429), Eta

(B.1.525), Gamma (P.1, P.1.1, P.1.2), Iota (B.1.526), Kappa (B.1.617.1), Lambda (C.37), Mu (B.1.621), Omicron (B.1.1.529, BA.2, BA.4, BA4.6, BA.5, BQ.1, BQ.1.1, XBB.1.5), and Zeta (P.2).

In some embodiments, the antigen is selected from or derived from the group consisting of rotavirus, foot and mouth disease virus, influenza A virus, influenza B virus, influenza C virus, H1N1, H2N2, H3N2, H5N1, H7N7, H1N2, H9N2, H7N2, H7N3, H1N7, human parainfluenza type 2, herpes simplex virus, Epstein-Barr virus, varicella virus, porcine herpesvirus 1, cytomegalovirus, lyssavirus, Bacillus anthracis, poliovirus, Hepatitis A, Hepatitis B, Hepatitis C, Hepatitis E, distemper virus, Venezuelan equine encephalomyelitis, feline leukemia virus, reovirus, respiratory syncytial virus, Lassa fever virus, polyoma tumor virus, canine parvovirus, papilloma virus, tick borne encephalitis virus, rinderpest virus, human rhinovirus species, Enterovirus species, Mengovirus, paramyxovirus, avian infectious bronchitis virus, human T-cell leukemia-lymphoma virus 1, human immunodeficiency virus-1, human immunodeficiency virus-2, lymphocytic choriomeningitis virus, parvovirus B19, adenovirus, rubella virus, yellow fever virus, dengue virus, bovine respiratory syncytial virus, corona virus, Bordetella pertussis, Bordetella bronchiseptica, Bordetella parapertussis, Brucella abortis, Brucella melitensis, Brucella suis, Brucella ovis, Brucella species, Escherichia coli, Salmonella species, Salmonella typhi, Streptococci, Vibrio cholera, Vibrio parahaemolyticus, Shigella, Pseudomonas, tuberculosis, avium, Bacille Calmette Guerin, Mycobacterium leprae, Pneumococci, Staphlylococci, Enterobacter species, Rochalimaia henselae, Pasteurella haemolytica, Pasteurella multocida, Chlamydia trachomatis, Chlamydia psittaci, Lymphogranuloma venereum, Treponema pallidum, Haemophilus species, Mycoplasma bovigenitalium, Mycoplasma pulmonis, Mycoplasma species, Borrelia burgdorferi, Legionalla pneumophila, Colstridium botulinum, Corynebacterium diphtheriae, Yersinia entercolitica, Yersinia pestis, Rickettsia rickettsii, Rickettsia typhi, Rickettsia prowsaekii, Ehrlichia chaffeensis, Anaplasma phagocytophilum, Plasmodium falciparum, Plasmodium vivax, Plasmodium malariae, Schistosomes, trypanosomes, Leishmania species, Filarial nematodes, trichomoniasis, sarcosporidiasis, Taenia saginata, Taenia solium, Leishmania, Toxoplasma gondii, Trichinella spiralis, coccidiosis, Eimeria tenella, Cryptococcus neoformans, Candida albican, Apergillus fumigatus, coccidioidomycosis, Neisseria gonorrhoeae, malaria circumsporozoite protein, malaria merozoite protein, trypanosome surface antigen protein, pertussis, alphaviruses, adenovirus, diphtheria toxoid, tetanus toxoid, meningococcal outer membrane protein, streptococcal M protein, Influenza hemagglutinin, cancer antigen, tumor antigens, toxins, Clostridium perfringens epsilon toxin, ricin toxin, pseudomonas exotoxin, exotoxins, neurotoxins, cytokines, cytokine receptors, monokines, monokine receptors, plant pollens, animal dander, and dust mites.

Other antigens associated with various diseases and infections are known in the art. It is contemplated that any antigen may be used in the methods and compositions described herein.

In some embodiments, the one or more antigens of the antigenic payload are present in the particles in an amount of about 0.1 wt. % to about 50 wt. %, about 0.5 wt. % to about 45 wt. %, about 1 wt. % to about 40 wt. %, about 1 wt. % to about 35 wt. %, about 1 wt. % to about 30 wt. %, about 1 wt. % to about 25 wt. %, about 1 wt. % to about 20 wt. %, about 1 wt. % to about 15 wt. %, about 1 wt. % to about 10 wt. %, about 1 wt. % to about 5 wt. %, or any amount or range therein. In some embodiments, the one or more antigens of the antigenic payload are present in the particles in an amount of about 0.1 wt. % to about 20 wt. %, about 0.5 wt. % to about 18 wt. %, about 1 wt. % to about 15 wt. %, about 1 wt. % to about 10 wt. %, about 1 wt. % to about 5 wt. %, about 1 wt. % to about 3 wt. %, or any amount or range therein. In other embodiments, the one or more antigens of the antigenic payload are present in the particles in an amount of about 0.1 wt. % to about 10 wt. %, about 0.1 wt. % to about 9 wt. %, about 0.1 wt. % to about 8 wt. %, about 0.1 wt. % to about 7 wt. %, about 0.1 wt. % to about 6 wt. %, about 0.1 wt. % to about 5 wt. %, about 0.1 wt. % to about 4 wt. %, about 0.1 wt. % to about 3 wt. %, about 0.1 wt. % to about 2 wt. %, about 0.1 wt. % to about 1 wt. %, or about 5 wt. % to about 15 wt. %, about 7 wt. % to about 13 wt. %, or about 9 wt. % to about 11 wt. %. In other embodiments, the one or more antigens are present in the particles in an amount of about 10 wt. % to about 50 wt. %, about 15 wt. % to about 45 wt. %, about 20 wt. % to about 40 wt. %, or about 25 wt. % to about 35 wt. % In other embodiments, the one or more antigens of the antigenic payload are present in the particles in an amount of about 0.1 wt. %, about 0.5 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, about 15 wt. %, about 15.5 wt. %, about 16 wt. %, about 16.5 wt. %, about 17 wt. %, about 17.5 wt. %, about 18 wt. %, about 18.5 wt. %, about 19 wt. %, 19.5 wt. %, or about 20 wt. %.

In some embodiments, the non-antigenic payload comprises one or more of an adjuvant, a protein, peptide, nucleic acid, small molecule, pharmaceutical drug, bacteria, virus, and whole cell lysate.

Some embodiments may include one or more adjuvants. Specific examples of adjuvants include, but are not limited to, aluminum salts (such as aluminum hydroxide, aluminum phosphate, and aluminum sulfate), 3 de-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Publication No. WO2007/109812 to Sutton et al.), imidazoquinoxaline compounds (see International Publication No. WO 2007/109813 to Sutton et al.) and saponins, such as QS21 (see Kensil et al., in Vaccine Design: The Subunit and Adjuvant Approach (eds. Powell & Newman, Plenum Press, NY, 1995); and U.S. Pat. No. 5,057,540 to Kensil et al.). In some embodiments, the adjuvant is Freund's adjuvant (complete or incomplete). Other adjuvants include oil-in-water emulsions (such as squalene or peanut oil), cholera toxin B subunit, TLR5 ligands such as flagellin, human papillomavirus L1 or L2 protein, herpes simplex glycoprotein D (gD), complement C4 binding protein, TLR4 ligands such as MPL, and IL-1ß, lysolecithin, pluronic polyols, polyanions, dinitrophenol, iscomatrix, liposome polycation DNA particles, TLR7 ligands such as imiquimod, TLR7/8 ligands such as resiquimod (R848), TLR9 ligands such as CpG polynucleotides, and Stimulator of Interferon Genes (STING) activating adjuvants.

Furthermore, adjuvants and specific examples of adjuvants that can be included in the particles are provided in the Table 1 below.

TABLE 1
Adjuvant Examples

| Adjuvant Class | Examples | |
|---|---|---|
| Liposomes | AS01 | Virus-like particles |
| | Virosomes | |
| Mineral Salts | Aluminum hydroxide | Calcium phosphate |
| | Aluminum phosphate | |
| Oil Emulsions | MF59 | AS03 |
| | AS02 | |
| Polymers | Polyanhydrides (CPH, CPP, SA, CPTEG) | Poly(vinyl alcohol) |
| | Polysulfones | Poly(caprolactone) |
| | Polyesters (PLGA, PLA, PGA, PCL) | Poly(hydroxybutyrate) |
| | Micelles (PDEAEM, Pluronic F127) | Chitosan |
| | Polyethers (PEG) | |
| Polysaccharides | β-glucans | Mannose |
| Saponins | Quillaja saponins | QS-21 |
| | Quill A | ISCOMs |
| STING-activating | Cyclic dinucleotides (CDNs) | R,R-CDG |
| TLR Agonists | CpG Oligodeoxynucleotide | MPLA |
| | Poly I:C | Flagellin |
| | Imiquimod/Resiquimod | AS04 |

In some embodiments, the adjuvant may include one or more of a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, cyclic dinucleotides, CpG oligodeoxynucleotide (CpG ODN), and a Stimulator of Interferon Genes (STING) activating adjuvant.

In some embodiments, the Toll-like receptor (TLR) agonist comprises one or more of triacylated lipoproteins, lipoteichoic acid, peptidoglycans, Zymosan, Pam3CSK4, Diacylated lipopeptides, HSPs, HMGB1, uric acid, fibronectin, ECM proteins, double stranded RNA, poly I: C, lipopolysaccharides, B-defensin, 2, fibronectin EDA, HMGB1, tenascin C, flagellin, single stranded RNA, CpG-A, poly G10, poly G3, and unmethylated CpG DNA. Examples of TLR agonists are described in, for example, Kaczanowska et al., J Leukoc Biol. 2013 June; 93 (6): 847-863.

In some embodiments, the adjuvant is a CpG ODN. In some embodiments, the CpG ODN is a class A CpG ODN, a class B CpG ODN, or a class C CpG ODN. In some embodiments, the CpG ODN adjuvant comprises one or more of CpG ODN 1018, CpG ODN 1585, CpG ODN 2216, CpG ODN 2336, CpG ODN 1668, CpG ODN 1826, CpG ODN 2006, CpG ODN 2007, CpG ODN BW006, CpG ODN D-SL01, CpG ODN 2395, CpG ODN M362, CpG ODN D-SL03. In some embodiments, the CpG ODN adjuvant is a CpG ODN 1668, CpG ODN 2006 (also known as CpG ODN 7909), CpG ODN 2007, or a combination thereof.

It is contemplated that one or more non-antigenic payloads may be entrapped or substantially entrapped within the interior of a particle. In some embodiments, the non-antigenic payload may be entrapped or substantially entrapped within the interior of a particle in an amount of about 0.1 wt. % to about 50 wt. %, about 0.5 wt. % to about 45 wt. %, about 1 wt. % to about 40 wt. %, about 1 wt. % to about 35 wt. %, about 1 wt. % to about 30 wt. %, about 1 wt. % to about 25 wt. %, about 1 wt. % to about 20 wt. %, about 1 wt. % to about 15 wt. %, about 1 wt. % to about 10 wt. %, about 1 wt. % to about 5 wt. %, or any amount or range therein.

In some embodiments, one or more non-antigenic payloads (e.g., an adjuvant) are present in the particles in an amount of about 0.1 wt. % to about 15 wt. %, about 0.5 wt. % to about 10 wt. %, about 0.5 wt. % to about 9 wt. %, about 0.5 wt. % to about 8 wt. %, about 0.5 wt. % to about 7 wt. %, about 0.5 wt. % to about 6 wt. %, about 0.5 wt. % to about 5 wt. %, about 0.5 wt. % to about 5 wt. %, about 0.5 wt. % to about 4 wt. %, about 0.5 wt. % to about 3 wt. %, about 0.5 wt. % to about 3 wt. %, about 0.5 wt. % to about 2 wt. %, or about 0.5 wt. % to about 1 wt. %, or any amount or range therein. In other embodiments, the one or more non-antigenic payloads are present in the particles in an amount of about 0.1 wt. % to about 10 wt. %, about 0.1 wt. % to about 9 wt. %, about 0.1 wt. % to about 8 wt. %, about 0.1 wt. % to about 7 wt. %, about 0.1 wt. % to about 6 wt. %, about 0.1 wt. % to about 5 wt. %, about 0.1 wt. % to about 4 wt. %, about 0.1 wt. % to about 3 wt. %, about 0.1 wt. % to about 2 wt. %, or about 0.1 wt. % to about 1 wt. %, or any amount or range therein.

In other embodiments, the non-antigenic payload is present in the particles in an amount of about 0.1 wt. %, about 0.2 wt. %, about 0.3 wt. %, about 0.4 wt. %, about 0.5 wt. %, 0.6 wt. %, about 0.7 wt. %, about 0.8 wt. %, about 0.9 wt. %, about 1 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, or about 10 wt. %.

In some embodiments, substantially all of the antigenic and/or non-antigenic payloads are entrapped within the interior of the particle.

In some embodiments, a composition comprises particles comprising a material soluble in a solvent suitable for spray drying; and one or more antigenic and/or non-antigenic payloads are entrapped within the particles.

In some embodiments, a composition comprises particles comprising one or more polymers, wherein the polymers comprise one or more of sebacic anhydride (SA), 1,6-bis(p-carboxyphenoxy)hexane (CPH), 1,8-bis(p-carboxyphenoxy)-3,6-dioxaoctane (CPTEG), CPTEG:CPH copolymer, PGLA, and a biocompatible polymer other than a polyanhydride that is soluble in a solvent suitable for spray drying; and one or more antigenic and/or non-antigenic payloads are entrapped within the particles.

In some embodiments, the particles comprise a polyanhydride, and the particles are stable at room temperature for at least 6 months, at least 9 months, at least 12 months, at least 15 months, at least 18 months, at least 21 months, at least 24 months, at least 27 months, at least 30 months, at least 33 months, at least 36 months, or greater than 36 months. In some embodiments, the particles are stable at room temperature for at least 3 years, at least 4 years, at least 5 years, at least 6 years, at least 7 years, at least 8 years, at least 9 years, at least 10 years, or greater than 10 years. Preferably, the structural stability of the antigen and antigenicity of the antigen, when examined, is the same or substantially the same at the time of testing (e.g., at month 6, 12, 36, etc.) compared to month the time of forming the particles as determined by one or more of SDS-PAGE, circular dichroism, and ELISA; and/or a particle size of the particles and morphology of the particles, when examined, is the same or substantially the same at the time of testing (e.g., at month 6, 12, 36, etc.) compared to the time of forming the particles as determined by dynamic light scattering and/or electron microscopy (e.g., scanning electron microscopy). The thermal stability of the particles also may be examined by administering to a subject the particles or compositions comprising the same and measuring the immune response to the particles. For example, the particles may be administered to a subject and the amount of serum antigen-specific antibodies induced measured via ELISA. Particles or compositions comprising the same are considered thermally stable over an indicated time period if the immune response is the same or substantially the same to the immune response elicited as at the time of forming the particles.

Accordingly, in some embodiments, a composition comprises particles comprising one or more polymers, wherein the polymers comprise one or more of sebacic anhydride (SA), 1,6-bis(p-carboxyphenoxy)hexane (CPH), 1,8-bis(p-carboxyphenoxy)-3,6-dioxaoctane (CPTEG), and a CPTEG:CPH copolymer, and one or more antigenic and/or non-antigenic payloads are entrapped within the particles. In some embodiments, the polymers comprise 20:80 CPTEG:CPH, PolySA, 10:90 CPTEG:SA, or 20:80 CPH:SA.

In other embodiments, a composition comprises particles comprising one or more polyanhydride polymers, wherein the polyanhydride polymers comprise one or more of sebacic anhydride (SA), 1,6-bis(p-carboxyphenoxy)hexane (CPH), 1,8-bis(p-carboxyphenoxy)-3,6-dioxaoctane (CPTEG), and a CPTEG:CPH copolymer; an antigenic payload entrapped within the particles, wherein the particles are stable at room temperature for at least six months, and wherein the polymers have an average molecular weight of about 5,000 g/mol to about 30,000 g/mol. Optionally, the compositions may include one or more non-antigenic payloads (e.g., an adjuvant) either co-entrapped in the particle with the antigen, or free in the composition, or both.

In other embodiments, a composition comprises particles comprising one or more polyanhydride polymers, wherein the polyanhydride polymers comprise one or more of sebacic anhydride (SA), 1,6-bis(p-carboxyphenoxy)hexane (CPH), 1,8-bis(p-carboxyphenoxy)-3,6-dioxaoctane (CPTEG), and a CPTEG:CPH copolymer; and an antigenic and/or non-antigenic payload entrapped within the particles, wherein the particles are stable at room temperature for at least six months, and wherein the polymers have an average molecular weight of about 7,000 g/mol to about 15,000 g/mol.

In other embodiments, a composition comprises particles comprising one or more polyanhydride polymers, wherein the polyanhydride polymers comprise one or more of sebacic anhydride (SA), 1,6-bis(p-carboxyphenoxy)hexane (CPH), 1,8-bis(p-carboxyphenoxy)-3,6-dioxaoctane (CPTEG), and a CPTEG:CPH copolymer; and an antigenic and/or non-antigenic payload entrapped within the particles, wherein the particles are stable at room temperature for at least six months, and wherein the polymers have an average molecular weight of about 10,000 g/mol to about 12,000 g/mol.

Preferably, the particles, antigens, and non-antigens described herein may be used to prepare therapeutic pharmaceutical compositions, for example, by combining the particles and adjuvants with a pharmaceutically acceptable diluent, excipient, or carrier. The particles may be added to a carrier in the form of a salt or solvate. Examples of pharmaceutically acceptable salts are organic acid addition salts formed with acids that form a physiologically acceptable anion, for example, tosylate, methanesulfonate, acetate, citrate, malonate, tartrate, succinate, benzoate, ascorbate, a-ketoglutarate, and y-glycerophosphate. Suitable inorganic salts also may be formed, including hydrochloride, halide, sulfate, nitrate, bicarbonate, and carbonate salts, sodium chloride, calcium chloride, sodium phosphate, monosodium glutamate, and aluminum salts (e.g., aluminum hydroxide, aluminum phosphate, alum (potassium aluminum sulfate), or a mixture of such aluminum salts.

Pharmaceutically acceptable salts may be obtained using standard procedures well known in the art, for example by reacting a sufficiently basic compound such as an amine with a suitable acid to provide a physiologically acceptable ionic compound. Alkali metal (for example, sodium, potassium or lithium) or alkaline earth metal (for example, calcium) salts of carboxylic acids can also be prepared by analogous methods.

Immunogenic compositions (such as a vaccine) may be formulated and administered in accordance with standard techniques well known to those skilled in the art. For example, a vaccine can be prepared by any suitable method, such as the methods described by Franchini et al., (International Patent Publication PCT/US2003/035499), Kipper et al., (U.S. Pat. No. 7,858,093), Narasimhan et al., (U.S. Pat. No. 10,874,737), or Caputa et al. (U.S. Pat. No. 5,554,371, for example, at col. 7). Other useful techniques can be used such as those described by Cleary (U.S. Pat. No. 5,846,547, for example, at col. 6-7).

The disclosure also provides for methods of preparing particles and compositions comprising the particles. For example, a method for manufacturing particle-based composition comprises the steps of i) reducing a lyophilized antigenic and/or a non-antigenic payload to an average size of about 10 nanometers to about 1500 nanometers in the presence of a first solvent to form a payload suspension; ii) mixing the payload suspension with a solution to form a mixture, wherein the solution comprises a material soluble in a second solvent, wherein the second solvent comprises a solvent suitable for spray drying; and iii) spray drying the mixture to form the particle-based composition.

The disclosure also provides for methods of preparing particles and compositions comprising the particles. For example, a method for manufacturing particle-based composition comprises the steps of i) spray drying the antigenic and/or a non-antigenic payload to an average size of about 10 nanometers to about 1500 nanometers in the presence of a first solvent to form a payload suspension; ii) mixing the payload suspension with a solution to form a mixture, wherein the solution comprises a material soluble in a second solvent, wherein the second solvent comprises a solvent suitable for spray drying; and iii) spray drying the mixture to form the particle-based composition.

Suitable methods for reducing the lyophilized the antigenic and/or non-antigenic payload to an average size of about 10 nanometers to about 1500 nanometers comprises milling or grinding the lyophilized antigenic or the non-antigenic payload. Preferably, the milling comprises wet milling. In some embodiments, the milling or the grinding comprises a use of a grinding media. In some embodiments, the methods for preparing the antigenic and/or non-antigenic payload comprises spray drying the dissolved payload to form a dry powder with an average size of about 10 nanometers to about 1500 nanometers, which may then be suspended in the material solution.

Alternative suitable methods for reducing the lyophilized antigenic and/or non-antigenic payload to an average size of about 10 nanometers to about 1500 nanometers may comprise high pressure homogenization, sonication, precipitation, emulsification, and cryogenic milling or grinding.

In some embodiments, the first solvent and the second solvent are the same or different solvents. In some embodiments, the first solvent comprises any solvent suitable reducing the size of the antigenic or the non-antigen payloads. In some embodiments, the first solvent comprises any solvent suitable for milling or grinding the antigenic or the non-antigen payloads and the second solvent comprises any solvent suitable for dissolving the materials for spray drying the particles.

In some embodiments, the first solvent is suitable to suspend the antigenic and/or the non-antigenic payload, and comprises one or more of an alcohol, a halogenated hydrocarbon, an aliphatic hydrocarbon, an aromatic hydrocarbon, a ketone, an ester, an ether, a nitrile, an amide, and acid, and a base. In some embodiments, the first solvent comprises one or more of ethanol, chloroform, acetone, dichloromethane, acetonitrile, ethyl acetate, isopropanol, acetic acid, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene. In some embodiments, the first solvent comprises one or more of ethanol, chloroform, acetone, dichloromethane, acetonitrile, ethyl acetate, and isopropanol. In some embodiments, the alcohol is ethanol.

In some embodiments, the second solvent comprises one or more of an alcohol, a halogenated hydrocarbon, an aliphatic hydrocarbon, an aromatic hydrocarbon, a ketone, an ester, an ether, a nitrile, an amide, and acid, and a base. In some embodiments, the second solvent comprises one or more of chloroform, dichloromethane, ethanol, acetone, acetonitrile, ethyl acetate, isopropanol, acetic acid, acetonitrile, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene. In some embodiments, the halogenated hydrocarbon is a fluorinated hydrocarbon, a chlorinated hydrocarbon, a brominated hydrocarbon, or an iodinated hydrocarbon. In some embodiments, the second solvent comprises one or more of chloroform, dichloromethane, ethanol, acetone, acetonitrile, ethyl acetate, and isopropanol.

In some embodiments, the first solvent is an alcohol and the second solvent is a chlorinated hydrocarbon. In some embodiments, the first solvent is ethanol and the second solvent is dichloromethane or chloroform.

In some embodiments, the concentration of the antigen or the non-antigenic payload in the payload suspension is about 1 mg/mL to about 500 mg/mL, about 1 mg/mL to about 450 mg/mL, about 1 mg/mL to about 400 mg/mL, about 1 mg/mL to about 350 mg/mL, about 1 mg/mL to about 300 mg/mL, about 1 mg/mL to about 250 mg/mL, about 1 mg/mL to about 200 mg/mL, about 1 mg/mL to about 150 mg/mL, about 1 mg/mL to about 100 mg/mL, about 1 mg/mL to about 50 mg/mL, or about 1 mg/mL to about 25 mg/mL. In other embodiments, the concentration of the antigen or the non-antigenic payload in the milled suspension is about 5 mg/mL to about 50 mg/mL, about 5 mg/mL to about 30 mg/mL, about 5 mg/mL to about 20 mg/mL, about 5 mg/mL to about 15 mg/mL, or about 5 mg/mL to about 10 mg/mL.

In some embodiments, the concentration of the material in the mixture is about 0.1 mg/ml to about 500 mg/mL, about 0.1 mg/mL to about 450 mg/mL, about 0.1 mg/mL to about 400 mg/mL, about 0.1 mg/mL to about 350 mg/mL, about 0.1 mg/mL to about 300 mg/mL, about 0.1 mg/mL to about 250 mg/mL, about 0.1 mg/mL to about 200 mg/mL, about 0.1 mg/mL to about 150 mg/mL, about 0.1 mg/mL to about 100 mg/mL, about 0.1 mg/mL to about 50 mg/mL, about 0.1 mg/mL to about 25 mg/mL, about 0.1 mg/mL to about 20 mg/mL, about 0.1 mg/mL to about 15 mg/mL, about 0.1 mg/mL to about 10 mg/mL, or about 0.1 mg/mL to about 5 mg/mL. In some embodiments, the concentration of the material in the mixture is about 1 mg/mL to about 50 mg/mL, about 1 mg/mL to about 45 mg/mL, about 1 mg/mL to about 40 mg/mL, about 1 mg/ml to about 35 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, about 1 mg/mL to about 5 mg/mL, or about 1 mg/mL to about 2.5 mg/mL. In other embodiments, the concentration of the antigen or the non-antigenic payload in the milled suspension is about 5 mg/mL to about 50 mg/mL, about 5 mg/mL to about 30 mg/mL, about 5 mg/mL to about 20 mg/mL, about 5 mg/mL to about 15 mg/mL, or about 5 mg/mL to about 10 mg/mL.

In some embodiments, the material is a polymer, and the polymer is present in the mixture in an amount of about 0.1 mg/mL to about 500 mg/mL, about 0.1 mg/mL to about 450 mg/mL, about 0.1 mg/mL to about 400 mg/mL, about 0.1 mg/mL to about 350 mg/mL, about 0.1 mg/mL to about 300 mg/mL, about 0.1 mg/mL to about 250 mg/mL, about 0.1 mg/mL to about 200 mg/mL, about 0.1 mg/mL to about 150 mg/mL, about 0.1 mg/mL to about 100 mg/mL, about 0.1 mg/mL to about 50 mg/mL, about 0.1 mg/mL to about 25 mg/mL, about 0.1 mg/mL to about 20 mg/mL, about 0.1 mg/mL to about 15 mg/mL, about 0.1 mg/mL to about 10 mg/mL, or about 0.1 mg/mL to about 5 mg/mL. In some embodiments, the polymer is present in the mixture in an amount of about 1 mg/mL to about 50 mg/mL, about 1 mg/mL to about 45 mg/mL, about 1 mg/mL to about 40 mg/mL, about 1 mg/mL to about 35 mg/mL, about 1 mg/mL to about 30 mg/mL, about 1 mg/mL to about 25 mg/mL, about 1 mg/mL to about 20 mg/mL, about 1 mg/mL to about 15 mg/mL, about 1 mg/mL to about 10 mg/mL, about 1 mg/mL to about 5 mg/mL, or about 1 mg/mL to about 2.5 mg/mL. In other embodiments, the concentration of the antigen or the non-antigenic payload in the milled suspension is about 5 mg/mL to about 50 mg/mL, about 5 mg/mL to about 30 mg/mL, about 5 mg/mL to about 20 mg/mL, about 5 mg/mL to about 15 mg/mL, or about 5 mg/mL to about 10 mg/mL.

In some embodiments, at least 30% of the antigenic or the non-antigenic payload is entrapped within the particles, at least 40% of the antigenic or the non-antigenic payload is entrapped within the particles, at least 50% of the antigenic or the non-antigenic payload is entrapped within the particles, at least 60% of the antigenic or the non-antigenic payload is entrapped within the particles, at least 70% of the antigenic or the non-antigenic payload is entrapped within the particles, at least 80% of the antigenic or the non-antigenic payload is entrapped within the particles, at least 90% of the antigenic or the non-antigenic payload is entrapped within the particles, or at least 95% of the antigenic or the non-antigenic payload is entrapped within the particles. In some embodiments, at least 25% to about 90% of the antigenic or the non-antigenic payload is entrapped within the particles, at least 30% to about 80% of the antigenic or the non-antigenic payload is entrapped within the particles, at least 35% to about 70% of the antigenic or the non-antigenic payload is entrapped within the particles, at least 40% to about 60% of the antigenic or the non-antigenic payload is entrapped within the particles, or at least 45% to about 55% of the antigenic or the non-antigenic payload is entrapped within the particles.

In some embodiments, a method for manufacturing particle-based composition comprises the steps of i) grinding or milling a lyophilized antigenic and/or a non-antigenic payload to an average size of about 10 nanometers to about 1500 nanometers in the presence of a first solvent to form a payload suspension; ii) mixing the payload suspension with a solution to form a mixture, wherein the solution comprises a material soluble in a second solvent, wherein the second solvent comprises a solvent suitable for spray drying; and iii) spray drying the mixture to form the particle-based composition, wherein the particles have an average size of about 0.15 μm to about 10 μm.

In some embodiments, a method for manufacturing a particle-based composition comprises the steps of i) grinding or milling a lyophilized antigenic and/or a non-antigenic payload to an average size of about 10 nanometers to about 1500 nanometers in the presence of an alcohol to form a payload suspension; ii) mixing the payload suspension with a solution to form a mixture, wherein the solution comprises a material soluble in a chlorinated hydrocarbon suitable for spray drying; and iii) spray drying the mixture to form the particle-based composition, wherein the particles have an average size of about 0.15 μm to about 10 μm.

In some embodiments, a method for manufacturing a particle-based composition comprises the steps of i) wet milling a lyophilized antigenic and/or a non-antigenic payload to an average size of about 10 nanometers to about 1500 nanometers in the presence of ethanol to form a payload suspension; ii) mixing the payload suspension with a solution to form a mixture, wherein the solution comprises dichloromethane or chloroform; and iii) spray drying the mixture to form the particle-based composition, wherein the particles have an average size of about 0.15 μm to about 10 μm.

In some embodiments, a method for manufacturing particle-based composition comprises the steps of i) reducing a lyophilized antigenic and/or a non-antigenic payload to an average size of about 10 nanometers to about 1500 nanometers in the presence of a first solvent to form a payload suspension; ii) mixing the payload suspension with a solution to form a mixture, wherein the solution comprises a one or more polymers in a second solvent, wherein the polymers have an average molecular weight of about 5,000 g/mol to about 30,000 g/mol, and the second solvent comprises a solvent suitable for spray drying; and iii) spray drying the mixture to form the particle-based composition, wherein the particles have an average size of about 0.15 μm to about 10 μm.

In some embodiments, a method for manufacturing particle-based composition comprises the steps of i) grinding or milling a lyophilized antigenic and/or a non-antigenic payload to an average size of about 150 nanometers to about 1300 nanometers in the presence of a first solvent to form a payload suspension; ii) mixing the payload suspension with a solution to form a mixture, wherein the solution comprises a one or more polyanhydride polymers in a second solvent, wherein the polyanhydride polymers have an average molecular weight of about 7,000 g/mol to about 15,000 g/mol, and the second solvent comprises a solvent suitable for spray drying; and iii) spray drying the mixture to form the particle-based composition, wherein the particles have an average size of about 0.5 μm to about 1.5 μm.

Apparatuses and methods for spray drying compositions are known in the art, and are described, for example, in Ohtake et al., Vaccine, vol. 28, pp. 1275-1284 (2010) and Arpagaus et al., J. Pharm. Investig. 49, 405-426 (2019). Thus, in some embodiments, the spray drying step comprises the use of a spray dry apparatus, wherein the spray dry apparatus comprises an inlet temperature of about 10 degrees Celsius to about 120 degrees Celsius, about 10 degrees Celsius to about 100 degrees Celsius, about 10 degrees Celsius to about 90 degrees Celsius, about 10 degrees Celsius to about 80 degrees Celsius, about 10 degrees Celsius to about 70 degrees Celsius, or about 10 degrees Celsius to about 60 degrees Celsius.

In some embodiments, a method for manufacturing particle-based composition comprises the steps of i) high pressure homogenizing the antigenic or non-antigenic payload to an average size of about 10 nanometers to about 1500; ii) mixing the payload with a solution to form a mixture, wherein the solution comprises a one or more materials in a second solvent, and the second solvent comprises a solvent suitable for spray drying; and iii) spray drying the mixture to form the particle-based composition, wherein the particles have an average size of about 0.15 μm to about 10 μm.

In some embodiments, a method for manufacturing particle-based composition comprises the steps of i) sonicating the antigenic or non-antigenic payload to an average size of about 10 nanometers to about 1500; ii) mixing the payload with a solution to form a mixture, wherein the solution comprises a one or more materials in a second solvent, and the second solvent comprises a solvent suitable for spray drying; and iii) spray drying the mixture to form the particle-based composition, wherein the particles have an average size of about 0.15 μm to about 10 μm. In some embodiments, a method for manufacturing particle-based composition comprises the steps of i) precipitating the antigenic or non-antigenic payload in an anti-solvent to an average size of about 10 nanometers to about 1500; ii) mixing the payload with a solution to form a mixture, wherein the solution comprises a one or more materials in a second solvent, and the second solvent comprises a solvent suitable for spray drying; and iii) spray drying the mixture to form the particle-based composition, wherein the particles have an average size of about 0.15 μm to about 10 μm.

In some embodiments, a method for manufacturing particle-based composition comprises the steps of i) emulsifying the antigenic or non-antigenic payload to an average size of about 10 nanometers to about 1500; ii) mixing the payload with a solution to form a mixture, wherein the solution comprises a one or more materials in a second solvent, and the second solvent comprises a solvent suitable for spray drying; and iii) spray drying the mixture to form the particle-based composition, wherein the particles have an average size of about 0.15 μm to about 10 μm.

In some embodiments, a method for manufacturing particle-based composition comprises the steps of i) cryogenically milling the antigenic or non-antigenic payload to an average size of about 10 nanometers to about 1500; ii) mixing the payload with a solution to form a mixture, wherein the solution comprises a one or more materials in a second solvent, and the second solvent comprises a solvent suitable for spray drying; and iii) spray drying the mixture to form the particle-based composition, wherein the particles have an average size of about 0.15 μm to about 10 μm.

It is contemplated that any embodiment discussed in this specification may be implemented with respect to any method, reagent, or composition, and vice versa. Furthermore, the compositions may be used to achieve the methods of invention.

### Statement of Certain Embodiments of the Invention

1. In a first embodiment, a composition comprises particles comprising one or more materials soluble in a solvent suitable for spray drying; and one or more antigenic or non-antigenic payloads entrapped within the particles; wherein the one or more antigenic or non-antigenic payloads have an average size of about 10 nm to about 1500 nm, and the particles have an average size of about 0.15 μm to about 10 μm.

2. The composition of embodiment 1, wherein the one or more materials comprise one or more of a biodegradable material, a nonbiodegradable material, a chemically degradable material, a stimuli-responsive material, a water-soluble material, and a swellable material.

3. The composition of embodiment 1 or 2, wherein the biodegradable material comprises one or more of collagen, chitosan, alginate, hyaluronic acid, chondroitin sulfate, and gelatin.

4. The composition of any one of embodiments 1-3, wherein the materials comprise homopolymers or copolymers comprising one or more of polyanhydrides, polyesters, or another polymer soluble in a solvent suitable for spray drying.

5. The composition of any one of embodiments 1-4, wherein the biodegradable material comprises polyanhydrides, poly(lactic-co-glycolic acid) (PLGA), poly(lactic acid) (PLA), poly(glycolic acid) (PGA), polycaprolactone (PCL), poly(glycerol sebacate) (PGS), PEG-PCL, and polyhydroxyalkanoates (PHA).

6. The composition of any one of embodiments 1-5, wherein the biodegradable material comprises one or more of poly(N-isopropylacrylamide) (PNIPAAm) and poly(acrylic acid) (PAA).

7. The composition of any one of embodiments 1-6, wherein the non-biodegradable material comprises one or more of polyethylene (PE), poly(methyl methacrylate) (PMMA), polytetrafluoroethylene (PTFE), silicone, and polyurethane.

8. The composition of any one of embodiments 1-7, wherein the water swollen material comprises one or more of a pluronic comprising of hydrophilic poly(ethylene oxide) (PEO) and hydrophobic poly(propylene oxide) (PPO), poly(diethyl amino ethyl methacrylate) (PDEAEM), poly(ethylene glycol) (PEG), poly(vinyl alcohol) (PVA), poly(2-hydroxyethyl methacrylate) (PHEMA), and agarose.

9. The composition of any one of embodiments 1-8, wherein the material comprises homopolymers or copolymers comprising an average molecular weight of about 5,000 g/mol to about 30,000 g/mol.

10. The composition of any one of embodiments 1-9, wherein the material comprises homopolymers or copolymers comprising an average molecular weight of about 10,000 g/mol to about 25,000 g/mol.

11. The composition of any one of embodiments 1-10, wherein the material comprises homopolymers or copolymers comprising an average molecular weight of about 10,000 g/mol to about 12,000 g/mol.

12. The composition of any one of embodiments 1-11, wherein the material comprises homopolymers or copolymers comprising sebacic anhydride (SA), 1,6-bis(p-carboxyphenoxy)hexane (CPH), 1,8-bis(p-carboxyphenoxy)-3,6-dioxaoctane (CPTEG), or a CPTEG:CPH copolymer.

13. The composition of any one of embodiments 1-12, wherein the materials comprise: i) a CPTEG:CPH copolymer; ii) a CPTEG:SA copolymer; iii) a CPH:SA copolymer; iv) a CPTEG homopolymer; v) a CPH homopolymer; vi) a SA homopolymer; vii) a PLGA copolymer; viii) a PLA homopolymer; or ix) a PGA homopolymer.

14. The composition of any one of embodiments 1-13, wherein the materials comprise x) the CPTEG:CPH copolymer in a molar ratio of about 20:80; xi) the CPTEG:SA copolymer in a molar ratio of about 10:90; xii) the CPH:SA copolymer in a molar ratio of about 20:80; or xiii) the PLGA copolymer in a molar ratio of about 50:50.

15. The composition of any one of embodiments 1-14, wherein the antigen payload comprises a eukaryotic, prokaryotic, or viral antigen.

16. The composition of any one of embodiments 1-15, wherein the antigenic payload comprises a protein, peptide, nucleic acid, or small molecule.

17. The composition of any one of embodiments 1-16, wherein the antigenic payload comprises one or more of the antigen comprising one or more of influenza virus hemagglutinin (HA) protein, influenza virus nucleoprotein (NP), influenza virus neuraminidase (NA) protein, influenza matrix proteins (M), RSV pre- or post-fusion F protein, RSV G protein, or SARS-COV-2 spike protein(S), SARS-COV-2 nucleocapsid (N) protein, SARS-COV-2 membrane protein (M), SARS-COV-2 envelope (E) protein, Yersinia pestis fusion protein F1-V, and Bacillus anthracis protective antigen (PA).

18. The composition of any one of embodiments 1-17, wherein the non-antigenic payload comprises one or more of an adjuvant, a protein, peptide, nucleic acid, small molecule, pharmaceutical drug, bacteria, virus, and whole cell lysate.

19. The composition of any one of embodiments 1-18, wherein the non-antigenic payload comprises the adjuvant entrapped within the particles.

20. The composition of any one of embodiments 1-19, wherein the adjuvant is a CpG oligodeoxynucleotide (CpG ODN), a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, cyclic dinucleotides, and a Stimulator of Interferon Genes (STING) activating adjuvant.

21. The composition of any one of embodiments 1-20, wherein the antigenic and/or the non-antigenic payload is present in the particles in an amount of about 0.1 wt. % to about 50 wt. %.

22. The composition of any one of embodiments 1-21, wherein the antigenic and/or the non-antigenic payload is present in the particles in an amount of about 0.1 wt. % to about 45 wt. %.

23. The composition of any one of embodiments 1-22, wherein the antigenic and/or the non-antigenic payload is present in the particles in an amount of about 0.1 wt. % to about 40 wt. %.

24. The composition of any one of embodiments 1-23, wherein the antigenic and/or the non-antigenic payload is present in the particles in an amount of about 0.1 wt. % to about 35 wt. %.

25. The composition of any one of embodiments 1-24, wherein the antigenic and/or the non-antigenic payload is present in the particles in an amount of about 0.1 wt. % to about 30 wt. %.

26. The composition of any one of embodiments 1-25, wherein the antigenic and/or the non-antigenic payload is present in the particles in an amount of about 0.1 wt. % to about 25 wt. %.

27. The composition of any one of embodiments 1-26, wherein the antigenic and/or the non-antigenic payload is present in the particles in an amount of about 0.1 wt. % to about 20 wt. %.

28. The composition of any one of embodiments 1-27, wherein the antigenic and/or the non-antigenic payload is present in the particles in an amount of about 0.1 wt. % to about 15 wt. %.

29. The composition of any one of embodiments 1-28, wherein the antigenic or the non-antigenic payload is present in the particles in an amount of about 0.1 wt. % to about 10 wt. %.

30. The composition of any one of embodiments 1-29, wherein the composition comprises particles comprising one or more polymers, wherein the polymers comprise one or more of sebacic anhydride (SA), 1,6-bis(p-carboxyphenoxy)hexane (CPH), 1,8-bis(p-carboxyphenoxy)-3,6-dioxaoctane (CPTEG), and a CPTEG:CPH copolymer; one or more antigens entrapped within the particles, and wherein the one or more polymers comprises an average molecular weight of about 5,000 g/mol to about 30,000 g/mol.

31. The composition of any one of embodiments 1-30, wherein the composition comprises biocompatible particles comprising 1,8-bis(p-carboxyphenoxy)-3,6-dioxaoctane (CPTEG) and 1,6-bis(p-carboxyphenoxy)hexane (CPH) in a molar ratio of about 20:80; one or more adjuvants entrapped within the particle; one or more antigens entrapped within the particles; wherein the particles are stable at room temperature for at least six months; and wherein the 20:80 CPTEG:CPH polyanhydride polymer comprises an average molecular weight of about 5,000 g/mol to about 30,000 g/mol.

32. The composition of any one of embodiments 1-31, wherein the composition is stable at room temperature for at least 6 consecutive months, wherein the structural stability of the antigen, antigenicity of the antigen, particle size of the particles, and morphology of the particles is substantially the same following at least 6 months storage as compared to the time of formation of the particles as determined by one or more of gel electrophoresis, circular dichroism, fluorescence microscopy, electron microscopy, ELISA or other antibody binding assay, or measurement of an immune response in a test subject.

33. The composition of any one of embodiments 1-32, wherein the composition is stable at room temperature for at least 12 consecutive months, wherein structural stability of the antigen, antigenicity of the antigen, particle size of the particles, and morphology of the particles are substantially the same at month 12 as compared to the time of formation of the particle as determined by one or more of gel electrophoresis, circular dichroism, fluorescence microscopy, electron microscopy, ELISA or other antibody binding assay, or measurement of an immune response in a test subject.

34. The composition of any one of embodiments 1-33, wherein the composition is stable at room temperature for at least 36 consecutive months, wherein the structural stability of the antigen, antigenicity of the antigen, particle size of the particles, and morphology of the particles is substantially the same following at least 36 months storage as compared to the time of formation of the particles as determined by one or more of gel electrophoresis, circular dichroism, fluorescence microscopy, electron microscopy, ELISA or other antibody binding assay, or measurement of an immune response in a test subject.

35. The composition of any one of embodiments 1-34, wherein the composition is stable at room temperature for at least 6 consecutive years, wherein the structural stability of the antigen, antigenicity of the antigen, particle size of the particles, and morphology of the particles is substantially the same following at least 6 years storage as compared to the time of formation of the particles as determined by one or more of gel electrophoresis, circular dichroism, fluorescence microscopy, electron microscopy, ELISA or other antibody binding assay, or measurement of an immune response in a test subject.

36. The composition of any one of embodiments 1-35, wherein the solvent suitable for spray drying comprises one or more of an alcohol, a halogenated hydrocarbon, an aliphatic hydrocarbon, an aromatic hydrocarbon, a ketone, an ester, an ether, a nitrile, an amide, and acid, and a base.

37. The composition of any one of embodiments 1-36, wherein the solvent suitable for spray drying comprises one or more of chloroform, dichloromethane, ethanol, acetone, acetonitrile, ethyl acetate, isopropanol, acetic acid, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene.

38. The composition of any one of embodiments 1-37, wherein the solvent suitable for spray drying comprises one or more of chloroform, dichloromethane, ethanol, acetone, acetonitrile, ethyl acetate, and isopropanol.

39. The composition of any one of embodiments 1-38, wherein the size of the antigenic or non-antigenic payload is about 150 nm to about 1400 nm.

40. The composition of any one of embodiments 1-39, wherein the size of the antigenic or non-antigenic payload is about 200 nm to about 1300 nm.

41. The composition of any one of embodiments 1-40, wherein the size of the antigenic or non-antigenic payload is about 200 nm to about 1200 nm.

42. The composition of any one of embodiments 1-41, wherein the size of the antigenic or non-antigenic payload is about 400 nm to about 1200 nm.

43. The composition of any one of embodiments 1-42, wherein the size of the antigenic or non-antigenic payload is about 700 nm to about 1200 nm.

44. The composition of any one of embodiments 1-43, wherein the average size of the particle is about 0.2 μm to about 9 μm.

45. The composition of any one of embodiments 1-44, wherein the average size of the particle is about 0.2 μm to about 8 μm.

46. The composition of any one of embodiments 1-45, wherein the average size of the particle is about 0.3 μm to about 7 μm.

47. The composition of any one of embodiments 1-46, wherein the average size of the particle is about 0.4 μm to about 8 μm.

48. The composition of any one of embodiments 1-47, further comprising one or more pharmaceutically acceptable carrier, excipient, or diluent.

49. A method for manufacturing particle-based compositions comprising the steps of i) reducing a lyophilized antigenic or a non-antigenic payload to an average size of about 10 nanometers to about 1500 nanometers in the presence of a first solvent to form a payload suspension; ii) mixing the payload suspension with a solution to form a mixture, wherein the solution comprises a material and a second solvent, wherein the material is soluble in the second solvent, and the second solvent is suitable for spray drying; and iii) spray drying the mixture to form the particle-based composition.

50. The method of embodiment 49, wherein the reducing step comprises spray drying the antigenic or a non-antigenic payload.

51. The method of embodiment 49 or 50, wherein the reducing step comprises milling or grinding the lyophilized antigenic or the non-antigenic payload.

52. The method of embodiment 49-51, wherein the milling or the grinding comprises a use of a grinding media.

53. The method of any one of embodiments 49-52, wherein the milling comprises wet milling.

54. The method of any one of embodiments 49-53, wherein the first solvent comprises any solvent suitable for suspending and milling or grinding the antigenic or the non-antigen payloads; and wherein the second solvent comprises any solvent suitable for dissolving the polymer for spray drying.

55. The method of any one of embodiments 49-54, wherein the first solvent comprises one or more of an alcohol, a halogenated hydrocarbon, an aliphatic hydrocarbon, an aromatic hydrocarbon, a ketone, an ester, an ether, a nitrile, an amide, an acid, and a base.

56. The method of any one of embodiments 49-55, wherein the first solvent comprises one or more of ethanol, chloroform, acetone, dichloromethane, acetonitrile, ethyl acetate, isopropanol, acetic acid, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene.

57. The method of any one of embodiments 49-56, wherein the first solvent comprises one or more of ethanol, chloroform, acetone, dichloromethane, acetonitrile, ethyl acetate, and isopropanol.

58. The method of any one of embodiments 49-57, wherein the alcohol is ethanol.

59. The method of any one of embodiments 49-58, wherein the second solvent comprises one or more of an alcohol, a halogenated hydrocarbon, an aliphatic hydrocarbon, an aromatic hydrocarbon, a ketone, an ester, an ether, a nitrile, an amide, and acid, and a base.

60. The method of any one of embodiments 49-59, wherein the second solvent comprises one or more of chloroform, dichloromethane, ethanol, acetone, acetonitrile, ethyl acetate, isopropanol, acetic acid, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene.

61. The method of any one of embodiments 49-60, wherein the second solvent comprises one or more of chloroform, dichloromethane, ethanol, acetone, acetonitrile, ethyl acetate, and isopropanol.

62. The method of any one of embodiments 49-61, wherein the halogenated hydrocarbon is a fluorinated hydrocarbon, chlorinated hydrocarbon, a brominated hydrocarbon, or an iodinated hydrocarbon.

63. The method of any one of embodiments 49-62, wherein a concentration of antigen or non-antigenic payload in the payload suspension is about 0.1 mg/mL to about 500 mg/mL.

64. The method of any one of embodiments 49-63, wherein a concentration of antigen or non-antigenic payload in the payload suspension is about 0.1 mg/mL to about 450 mg/mL.

65. The method of any one of embodiments 49-64, wherein a concentration of antigen or non-antigenic payload in the payload suspension is about 0.1 mg/mL to about 400 mg/mL.

66. The method of any one of embodiments 49-65, wherein a concentration of antigen or non-antigenic payload in the payload suspension is about 0.1 mg/mL to about 350 mg/mL.

67. The method of any one of embodiments 49-66, wherein a concentration of antigen or non-antigenic payload in the payload suspension is about 0.1 mg/mL to about 300 mg/mL.

68. The method of any one of embodiments 49-67, wherein a concentration of antigen or non-antigenic payload in the payload suspension is about 0.1 mg/mL to about 250 mg/mL.

69. The method of any one of embodiments 49-68, wherein a concentration of antigen or non-antigenic payload in the payload suspension is about 0.1 mg/mL to about 200 mg/mL.

70. The method of any one of embodiments 49-69, wherein a concentration of antigen or non-antigenic payload in the payload suspension is about 0.1 mg/mL to about 150 mg/mL.

71. The method of any one of embodiments 49-70, wherein a concentration of antigen or non-antigenic payload in the payload suspension is about 0.1 mg/mL to about 100 mg/mL.

72. The method of any one of embodiments 49-71, wherein a concentration of antigen or non-antigenic payload in the payload suspension is about 0.1 mg/mL to about 75 mg/mL.

73. The method of any one of embodiments 49-72, wherein a concentration of antigen or non-antigenic payload in the payload suspension is about 0.1 mg/mL to about 50 mg/mL.

74. The method of any one of embodiments 49-73, wherein a concentration of antigen or non-antigenic payload in the payload suspension is about 0.1 mg/mL to about 25 mg/mL.

75. The method of any one of embodiments 49-74, wherein a concentration of antigen or non-antigenic payload in the payload suspension is about 0.1 mg/mL to about 20 mg/mL.

76. The method of any one of embodiments 49-75, wherein a concentration of the material in the mixture is about 0.1 mg/mL to about 500 mg/mL.

77. The method of any one of embodiments 49-76, wherein a concentration of the material in the mixture is about 0.1 mg/mL to about 450 mg/mL.

78. The method of any one of embodiments 49-77, wherein a concentration of the material in the mixture is about 0.1 mg/mL to about 400 mg/mL.

79. The method of any one of embodiments 49-78, wherein a concentration of the material in the mixture is about 0.1 mg/mL to about 350 mg/mL.

80. The method of any one of embodiments 49-79, wherein a concentration of the material in the mixture is about 0.1 mg/mL to about 300 mg/mL.

81. The method of any one of embodiments 49-80, wherein a concentration of the material in the mixture is about 0.1 mg/mL to about 250 mg/mL.

82. The method of any one of embodiments 49-81, wherein a concentration of the material in the mixture is about 0.1 mg/mL to about 200 mg/mL.

83. The method of any one of embodiments 49-82, wherein a concentration of the material in the mixture is about 0.1 mg/mL to about 150 mg/mL.

84. The method of any one of embodiments 49-83, wherein a concentration of the material in the mixture is about 0.1 mg/mL to about 100 mg/mL.

85. The method of any one of embodiments 49-84, wherein a concentration of the material in the mixture is about 0.1 mg/mL to about 50 mg/mL.

86. The method of any one of embodiments 49-85, wherein a concentration of the material in the mixture is about 0.1 mg/mL to about 25 mg/mL.

87. The method of any one of embodiments 49-86, wherein a concentration of the material in the mixture is about 0.1 mg/mL to about 10 mg/mL.

88. The method of any one of embodiments 49-87, wherein a concentration of the polymer in the mixture is about 0.1 mg/mL to about 500 mg/mL.

89. The method of any one of embodiments 49-88, wherein a concentration of the polymer in the mixture is about 0.1 mg/mL to about 450 mg/mL.

90. The method of any one of embodiments 49-89, wherein a concentration of the polymer in the mixture is about 0.1 mg/mL to about 400 mg/mL.

91. The method of any one of embodiments 49-90, wherein a concentration of the polymer in the mixture is about 0.1 mg/mL to about 350 mg/mL.

92. The method of any one of embodiments 49-91, wherein a concentration of the polymer in the mixture is about 0.1 mg/mL to about 300 mg/mL.

93. The method of any one of embodiments 49-92, wherein a concentration of the polymer in the mixture is about 0.1 mg/mL to about 250 mg/mL.

94. The method of any one of embodiments 49-93, wherein a concentration of the polymer in the mixture is about 0.1 mg/mL to about 200 mg/mL.

95. The method of any one of embodiments 49-94, wherein a concentration of the polymer in the mixture is about 0.1 mg/mL to about 150 mg/mL.

96. The method of any one of embodiments 49-95, wherein a concentration of the polymer in the mixture is about 0.1 mg/mL to about 100 mg/mL.

97. The method of any one of embodiments 49-96, wherein a concentration of the polymer in the mixture is about 0.1 mg/mL to about 50 mg/mL.

98. The method of any one of embodiments 49-97, wherein a concentration of the polymer in the mixture is about 0.1 mg/mL to about 25 mg/mL.

99. The method of any one of embodiments 49-98, wherein a concentration of the polymer in the mixture is about 0.1 mg/mL to about 15 mg/mL.

100. The method of any one of embodiments 49-99, wherein the antigen payload comprises a eukaryotic, prokaryotic, or viral antigen.

101. The method of any one of embodiments 49-100, wherein the antigenic payload comprises a protein, peptide, nucleic acid, or small molecule.

102. The method of any one of embodiments 49-101, wherein the antigenic payload comprises one or more of the antigen comprises Yersinia pestis fusion protein F1-V, Bacillus anthracis protective antigen (PA), influenza virus hemagglutinin (HA), influenza virus nucleoprotein (NP), influenza virus neuraminidase (NA), influenza matrix proteins (M), RSV pre- or post-fusion F protein, RSV G protein, or SARS-COV-2 spike protein(S), SARS-COV-2 nucleocapsid (N), SARS-CoV-2 membrane protein (M), and SARS-COV-2 envelope (E) protein.

103. The method of any one of embodiments 49-102, wherein the non-antigenic payload comprises one or more of an adjuvant, a protein, peptide, nucleic acid, small molecule, pharmaceutical drug, bacteria, virus, and a whole cell lysate.

104. The method of any one of embodiments 49-103, wherein the adjuvant is a CpG oligodeoxynucleotide (CpG ODN), a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, cyclic dinucleotides, and a Stimulator of Interferon Genes (STING) activating adjuvant.

105. The method of any one of embodiments 49-104, wherein the non-antigenic payload comprises the adjuvant entrapped within the particles.

106. The method of any one of embodiments 49-105, wherein at least 30% of the antigenic or the non-antigenic payload is entrapped within the particles.

107. The method of any one of embodiments 49-106, wherein at least 40% of the antigenic or the non-antigenic payload is entrapped within the particles.

108. The method of any one of embodiments 49-107, wherein at least 50% of the antigenic or the non-antigenic payload is entrapped within the particles.

109. The method of any one of embodiments 49-108, wherein at least 60% of the antigenic or the non-antigenic payload is entrapped within the particles.

110. The method of any one of embodiments 49-109, wherein at least 70% of the antigenic or the non-antigenic payload is entrapped within the particles.

111. The method of any one of embodiments 49-110, wherein at least 80% of the antigenic or the non-antigenic payload is entrapped within the particles.

112. The method of any one of embodiments 49-111, wherein at least 90% of the antigenic or the non-antigenic payload is entrapped within the particles.

113. The method of any one of embodiments 49-112, wherein at least 95% of the antigenic or the non-antigenic payload is entrapped within the particles.

114. The method of any one of embodiments 49-113, wherein the antigenic and/or the non-antigenic payload is present in the particles in an amount of about 0.1 wt. % to about 50 wt. %.

115. The method of any one of embodiments 49-114, wherein the antigenic and/or the non-antigenic payload is present in the particles in an amount of about 0.1 wt. % to about 45 wt. %.

116. The method of any one of embodiments 49-115, wherein the antigenic and/or the non-antigenic payload is present in the particles in an amount of about 0.1 wt. % to about 40 wt. %.

117. The method of any one of embodiments 49-116, wherein the antigenic and/or the non-antigenic payload is present in the particles in an amount of about 0.1 wt. % to about 35 wt. %.

118. The method of any one of embodiments 49-117, wherein the antigenic and/or the non-antigenic payload is present in the particles in an amount of about 0.1 wt. % to about 30 wt. %.

119. The method of any one of embodiments 49-118, wherein the antigenic and/or the non-antigenic payload is present in the particles in an amount of about 0.1 wt. % to about 25 wt. %.

120. The method of any one of embodiments 49-119, wherein the antigenic and/or the non-antigenic payload is present in the particles in an amount of about 0.1 wt. % to about 15 wt. %.

121. The method of any one of embodiments 49-120, wherein the antigenic or the non-antigenic payload is present in the particles in an amount of about 1 wt. % to about 10 wt. %.

122. The method of any one of embodiments 49-121, wherein the material comprises a polymer, wherein the polymer comprises one or more of sebacic anhydride (SA), 1,6-bis(p-carboxyphenoxy)hexane (CPH), 1,8-bis(p-carboxyphenoxy)-3,6-dioxaoctane (CPTEG), a CPTEG:CPH copolymer, and a polymer other than a polyanhydride that is soluble in a solvent suitable for spray drying.

123. The method of any one of embodiments 49-122, wherein the spray drying step comprises the use of a spray dry apparatus, wherein the spray dry apparatus comprises an inlet temperature of about 10 degrees Celsius to about 120 degrees Celsius.

124. The method of any one of embodiments 49-123, wherein the material comprises one or more of lipids, sugars and/or carbohydrates, proteins, salts, ionic compounds, silicon, metals and metal oxides, ceramics, amino acids, surfactants, and waxes.

125. The method of any one of embodiments 49-124, wherein the material comprises one or more of phospholipids, triglycerides, cholesterol, fatty acids, lactose, mannitol, trehalose, sucrose, dextrose, albumin, gelatin, casein, sodium chloride, calcium phosphate, magnesium sulfate, silicon dioxide, gold, iron oxide (magnetite), titanium dioxide, hydroxyapatite, bioactive glass, leucine, glycine, arginine, sodium dodecyl sulfate (SDS), polysorbate, beeswax, carnauba wax, and paraffin wax.

126. The composition of any one of embodiments 1-48, wherein the material comprises one or more of lipids, sugars and/or carbohydrates, proteins, salts, ionic compounds, silicon, metals and metal oxides, ceramics, amino acids, surfactants, and waxes.

127. The composition of any one of embodiments 1-48 and 126 wherein the material comprises one or more of phospholipids, triglycerides, cholesterol, fatty acids, lactose, mannitol, trehalose, sucrose, dextrose, albumin, gelatin, casein, sodium chloride, calcium phosphate, magnesium sulfate, silicon dioxide, gold, iron oxide (magnetite), titanium dioxide, hydroxyapatite, bioactive glass, leucine, glycine, arginine, sodium dodecyl sulfate (SDS), polysorbate, beeswax, carnauba wax, and paraffin wax.

128. The composition of any one of embodiments, 1-48, 126, and 127, wherein the composition is an immunogenic composition.

129. A composition manufactured according to the method of any one of embodiments 49-125.

The following examples are intended to illustrate the above invention and should not be construed as to narrow its scope. One skilled in the art will readily recognize that the examples suggest many other ways in which the invention could be practiced. It should be understood that numerous variations and modifications may be made while remaining within the scope of the invention.

### EXAMPLES

### Example 1. Thermostable Polyanhydride Particle-Based Compositions

Disclosed are PBVs with improved shelf stability compared to current vaccines that can maintain particle physiochemical characteristics and retain stability of encapsulated payloads when stored on the shelf for extended duration (≥6 months). Also disclosed is a new scalable, two-step manufacturing process of payload reduction and encapsulation technology (PRET) for the synthesis of thermostable PBVs.

### Shelf-Life and Thermostability of Polyanhydride PBVs

The unique physiochemical and thermal properties of polyanhydride copolymers contribute significantly to the extended stability of PBVs at room temperature. In this example, two polyanhydride PBV formulations were stored at room temperature to demonstrate long-term (≥4 years) shelf stability without the cold chain, during which particle and encapsulated payload stability was maintained. The 20:80 CPTEG:CPH PBVs encapsulating 10% (w/w) Yersinia pestis fusion protein F1-V or 10% (w/w) B. anthracis PA antigen were synthesized via flash nanoprecipitation. Briefly, the polyanhydride copolymer was dissolved 20 mg/mL in methylene chloride. The lyophilized antigens were suspended in the polymer solution and sonicated for 30 s to ensure even distribution. The particles were then precipitated into chilled pentane and collected via filtration. The PBV formulations were placed at room temperature with desiccant for storage.

Over the course of at least 33 months, PBV samples were removed from storage and analyzed. Particle size and morphology were characterized via scanning electron microscopy (SEM; FEI Quanta-FEG 250, FEI, Hillsboro, OR). The SEM micrographs were analyzed using Image J (National Institutes of Health, Bethesda, MD) to determine the average particle diameter. To observe the stability of the encapsulated antigens, stored PBV samples were suspended in nanopure water to allow for antigen release. The supernatant was collected and the amount of released antigen quantified with a micro bicinchoninic assay (BCA). The primary, secondary, and tertiary structure of the released antigen was determined with SDS-PAGE, circular dichroism (Jasco J-715, Jasco Analytical Instruments, Easton, MD), and fluorescence spectroscopy (SpectraMax M3, Molecular Devices, Sunnyvale, CA), respectively. The antigenicity of the release antigen was determined via ELISA. Each sample of released antigen was compared to fresh antigen diluted to the same concentration.

### Thermostability of PBV Particles Following Extended, Room Temperature Storage

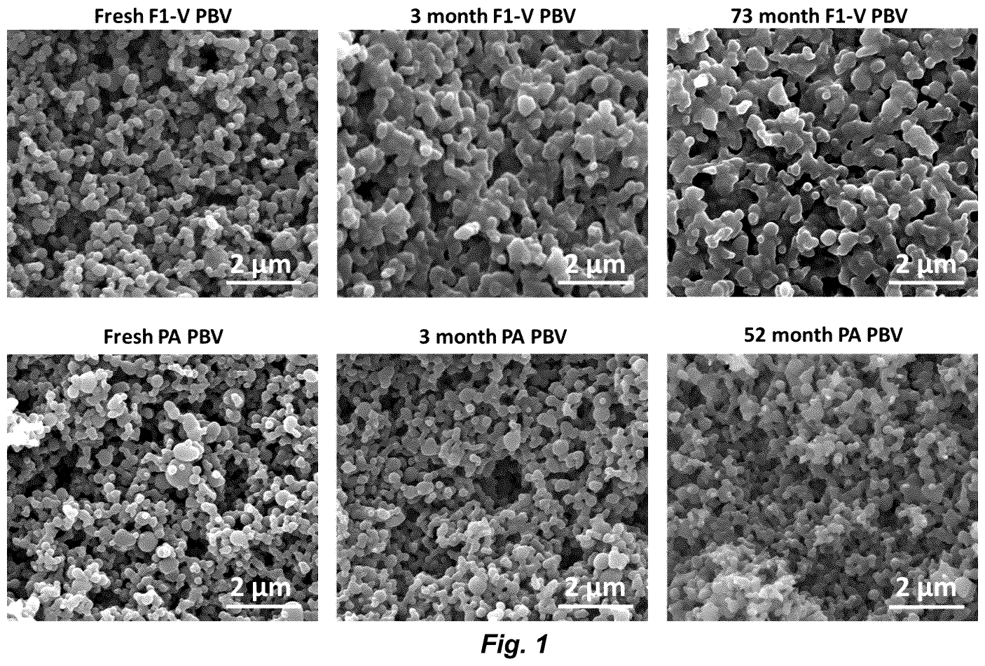

The micrographs of room temperature stored PBVs are shown in FIG. 1. Both F1-V and PA PBVs demonstrated semi-spherical particles that are partially aggregated, which are consistent with flash nanoprecipitation synthesis. No observable differences were noted in the morphology or aggregation of stored PBVs compared to those at Month 0 (freshly synthesized PBVs). Additionally, the diameters of the PBVs were found to be approximately 200-300 nm (n=200 particles), which was maintained at all time points evaluated.

Thermostability of Encapsulated Antigens after PBV Room Temperature Storage

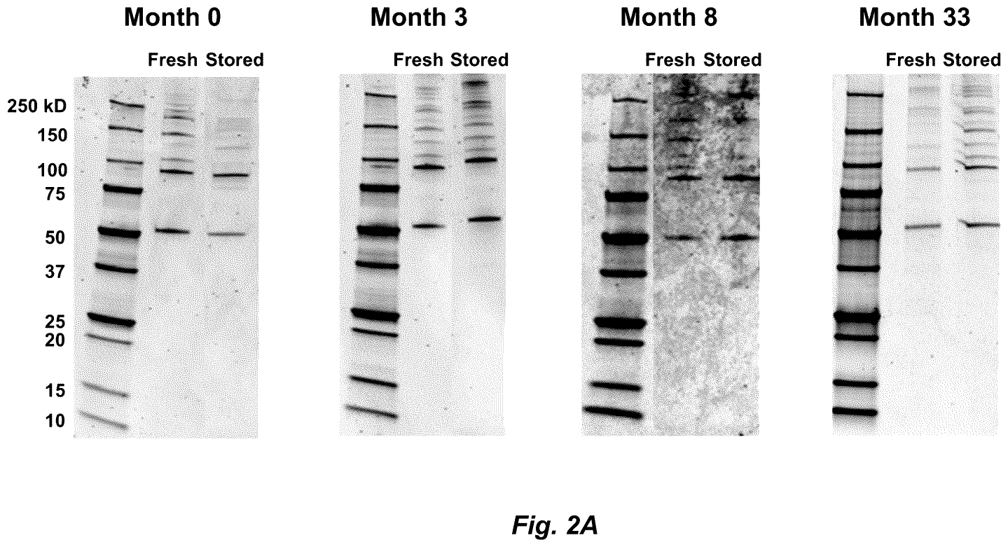

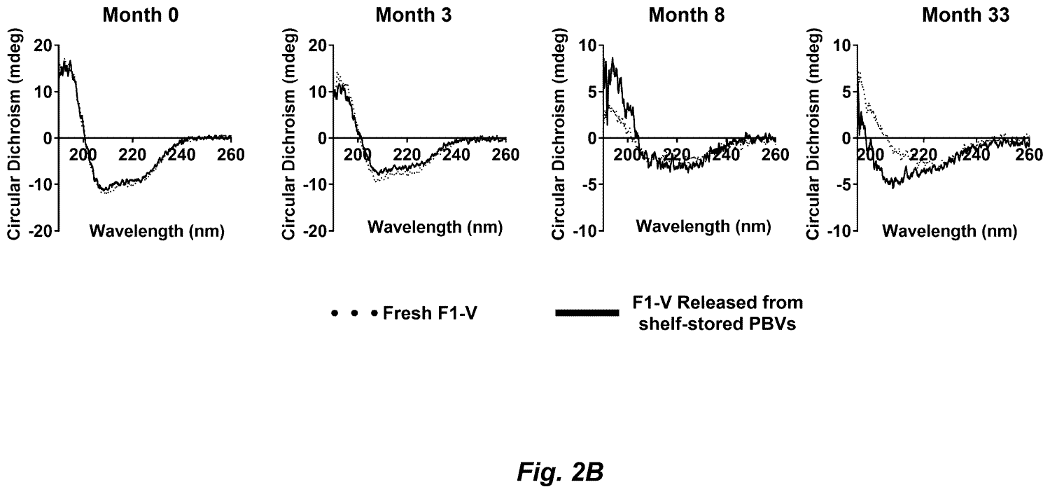

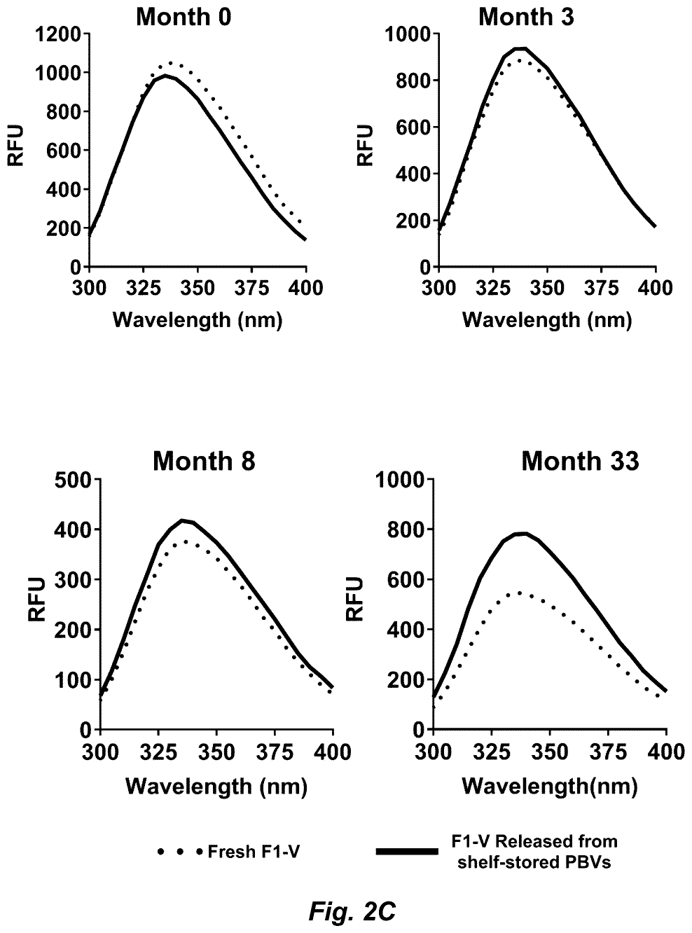

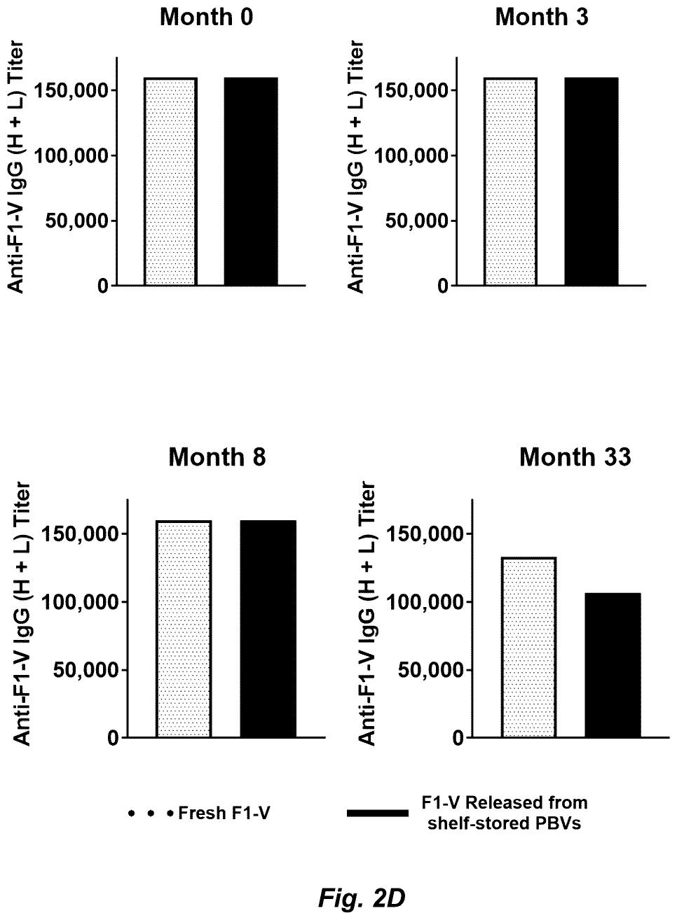

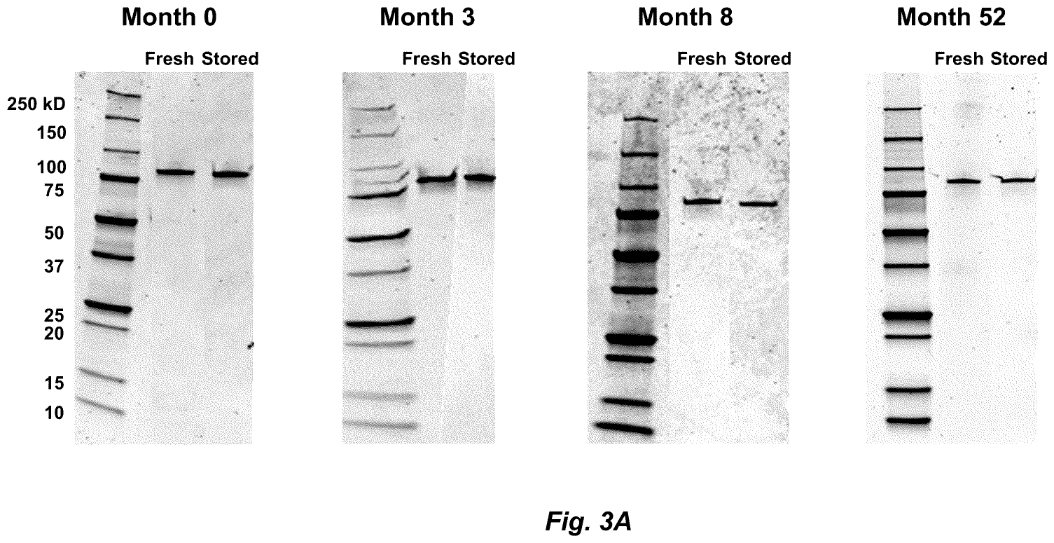

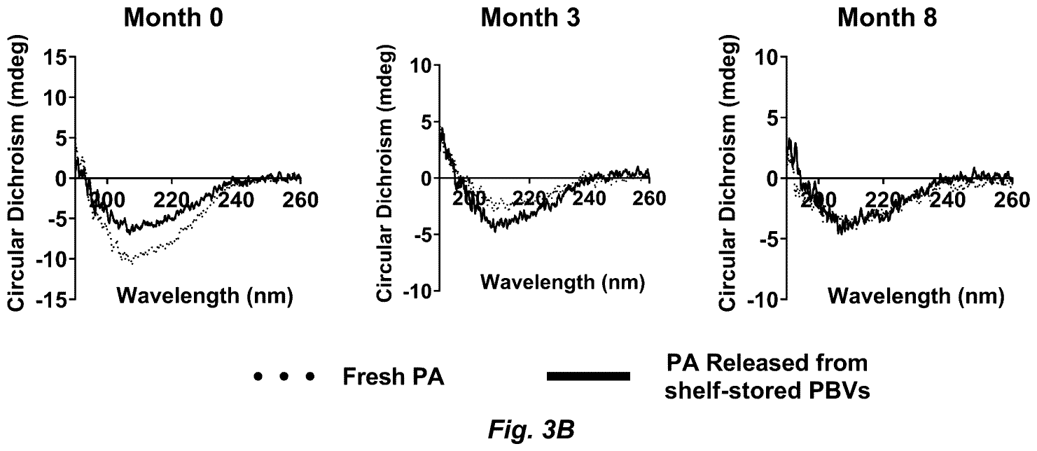

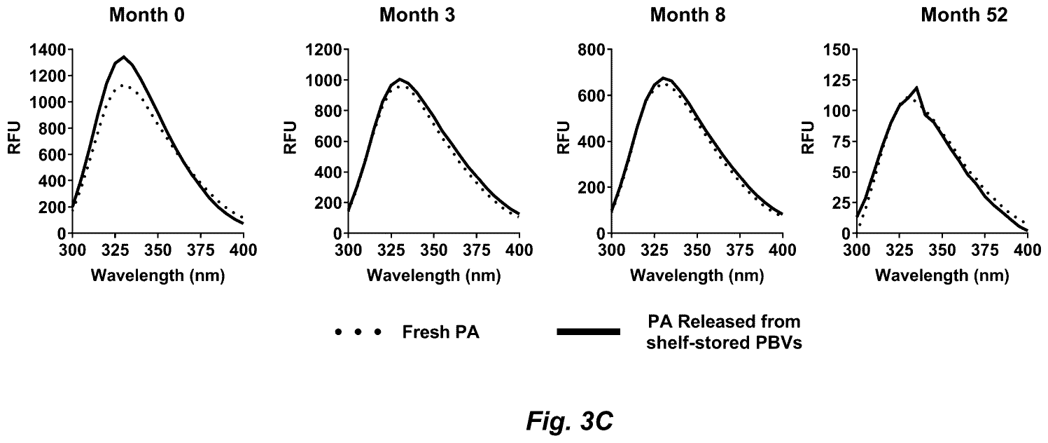

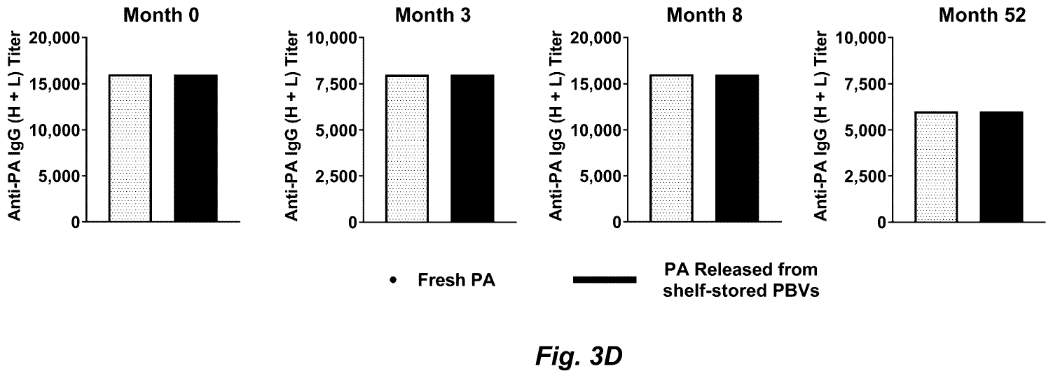

The encapsulated F1-V and PA antigens were also evaluated to determine maintenance of protein structural stability and antigenicity following storage of the PBV formulations at room temperature. Antigens were released from stored PBVs for 2-18 hours in phosphate buffered saline (PBS). A microBCA assay was performed to quantify the amount of released antigen, and fresh antigen controls of the same concentration were prepared. SDS-PAGE demonstrated the retention of monomeric and dimeric F1-V (FIG. 2A) and monomeric PA (FIG. 3A) at all storage time points similar to a fresh antigen control. Circular dichroism (FIGS. 2B & 3B) and fluorescence spectroscopy (FIGS. 2C & 3C) showed the secondary and tertiary structures of each antigen being maintained after PBV storage for at least 33 months. Although the intensity of each spectrum may vary based on the concentration of released antigen, no differences were observed when compared to fresh antigen prepared to the same concentration. Finally, ELISA (FIGS. 2D & 3D) demonstrated the antigenicity of each protein was maintained and recognized by anti-F1-V and anti-PA antibodies, respectively.

Altogether, these results demonstrate the thermostability of polyanhydride PBV formulations when stored for extended periods of time (i.e., greater than 6 months) at room temperature. Analyses showed the polyanhydride particle size and morphology was maintained, and the encapsulated antigen structural and antigenic stability preserved after room temperature storage of PBVs.

### Example 2. Scalable, Manufacturing Process Using Payload Reduction and Encapsulation Technology (PRET) for the Synthesis of PBVs

A novel, two-step manufacturing process has been developed for the scalable synthesis of PBVs: Payload Reduction and Encapsulation Technology (PRET). The PRET process first uses either wet milling with a suitable grinding media to reduce the diameter of antigenic or non-antigenic payload aggregates to approximately 1500 nm or smaller, or alternatively spray drying to prepare the antigenic or non-antigenic payload to a diameter of approximately 1500 nm or smaller. Next, the prepared payload is encapsulated into particles via spray drying. The PRET method is payload agnostic and is widely applicable to any antigenic or non-antigenic payload (such as proteins, adjuvants, peptides, nucleic acids, and small molecule therapeutics), assuming the payload can be dried to a solid and stably wet milled or can be dissolved and stably spray dried. Additionally, a wide variety of materials (e.g., polyanhydrides, polyesters, etc.) may be used to encapsulate the payloads. Compared to laboratory scale particle synthesis with flash nanoprecipitation, PBVs synthesized with PRET typically have more reproducible release kinetics, increased encapsulation efficiencies, greater batch-to-batch reproducibility, and higher yields that increase with scale of production.

### Reduction of Payload Aggregate Size Via Wet Milling

In order to effectively mill payloads to approximately 1500 nm or less, the payloads first need to be dried to a solid. The lyophilization process is generally suitable for drying a wide variety of payloads including but not limited to protein antigens, peptides, nucleic acids, adjuvants, or small molecules. The dried payloads are next suspended in a solvent at approximately 0.1 mg/mL to 500 mg/mL with grinding media. The solvent may be any solvent which can create a suspension of the payload, maintains payload stability, and is miscible with the polymer solvent used in subsequent spray drying of particles. The type of grinding media, amount of grinding media, amount of solvent, temperature, milling time, and milling energy may be varied to optimize conditions for each individual payload; the primary consideration is to maintain payload stability (e.g., molecular structure, functional activity, conformational structure, immunogenicity) following the milling process. Finally, an advantage of this process is that it minimizes losses of potentially expensive payloads during milling, as the entire procedure occurs as an integrated one-pot batch process.

In this example, payloads were suspended in approximately 1 mL of solvent with zirconia-containing beads. The samples were milled for 18 to 40 h, 10 to 30 Hz at 4° C., depending on the particular payload. Following milling, sample size was measured with dynamic light scattering (Zetasizer ZS90, Malvern Panalytical Ltd, United Kingdom). Table 2 demonstrates the effective milling of payloads to sizes ranging from 170 nm to 1227 nm using a broad selection of payloads, including model proteins, clinically relevant vaccine antigens, peptides, nucleic acids, and small molecules. Table 3 additionally demonstrates the effective milling of a model protein (OVA) to the size of approximately 1500 nm or smaller, ranging in sizes of 383 nm to 1120 nm, using a broad selection of solvents, which demonstrates the wide applicability of the milling process. Additionally, reproducible results were achieved (Table 2, Batch 10-12) when using the same payload and milling conditions. Of note, different milling apparatuses milling the same payload (Table 2, Batch 1 vs. Batch 2) were able to reduce the payloads to similar size.

TABLE 2
Size of Milled Payloads

| | Batch | | | | St. |
|---|---|---|---|---|---|
| | | | | Size | Dev. |
| Payload | No. | Payload | Mill | (nm) | (nm) |
| Protein | 1 | Bovine serum albumin | A | 649.4 | 77.4 |
| | | (BSA) | | | |
| | 2 | Bovine serum albumin | B | 1066.5 | 84.1 |
| | | (BSA) | | | |
| | 3 | F1-V | A | 1091.9 | 210.6 |
| | 4 | H1N1 hemagglutinin | B | 1055.9 | 104.4 |
| | | (HA) | | | |
| | 5 | H1N1 nucleoprotein | B | 1227.0 | 29.2 |
| | | (NP) | | | |
| | 6 | H5N1 hemagglutinin | A | 1072.8 | 36.2 |
| | | trimer (HA3) | | | |
| | 7 | Lysozyme | A | 176.6 | 0.8 |
| | 8 | Lysozyme | B | 272.7 | 1.5 |
| | 9 | Ovalbumin (OVA) | A | 473.6 | 3.2 |
| | 10 | Ovalbumin (OVA) | B | 575.1 | 18.3 |
| | 11 | Ovalbumin (OVA) | B | 586.1 | 26.1 |
| | 12 | Ovalbumin (OVA) | B | 612.2 | 11.3 |
| | 13 | Protective antigen | A | 789.4 | 59.8 |
| | | (PA) | | | |
| | 14 | Sheep IgG | B | 790.1 | 52.9 |
| Peptide | 15 | SIINFEKL (SEQ ID | B | 240.1 | 12.4 |
| | | NO: 1) | | | |
| | 16 | EIYQAGST (SEQ ID | B | 169.8 | 1.9 |
| | | NO: 2) | | | |
| | 17 | YTWFHAIHVSGTNGT (SEQ | B | 478.7 | 19.6 |
| | | ID NO: 3) | | | |
| | 18 | LALLLLDRL (SEQ ID | B | 368.0 | 8.6 |
| | | NO: 4) | | | |
| Small | 19 | Cyclic-di-GMP | B | 368.8 | 6.2 |
| Molecule | 20 | CpG ODN 1668 | A | 418.0 | 12.7 |
| | 21 | CpG ODN 1668 | B | 1032.6 | 26.2 |
| | 22 | CpG ODN 2006 | A | 620.9 | 58.9 |
| | 23 | CpG ODN 2006 | B | 889.5 | 86.8 |
| | 24 | CpG ODN 2007 | A | 361.8 | 18.7 |
| | 25 | Doxycycline hyclate | B | 698.4 | 11.6 |

TABLE 3
Effect of Milling Solvent

| Milling Solvent | OVA Size (Mean ± SEM) | |
|---|---|---|
| Acetone | 413.9 ± 5.1 | nm |
| Acetonitrile | 388.7 ± 7.9 | nm |
| Chloroform | 1106.0 ± 27.9 | nm |
| Ethanol | 956.4 ± 6.0 | nm |
| Ethyl acetate | 781.6 ± 21.0 | nm |
| Isopropyl alcohol | 383.4 ± 9.7 | nm |
| Methylene chloride | 1120.2 ± 80.1 | nm |

### Stability of Milled Payloads

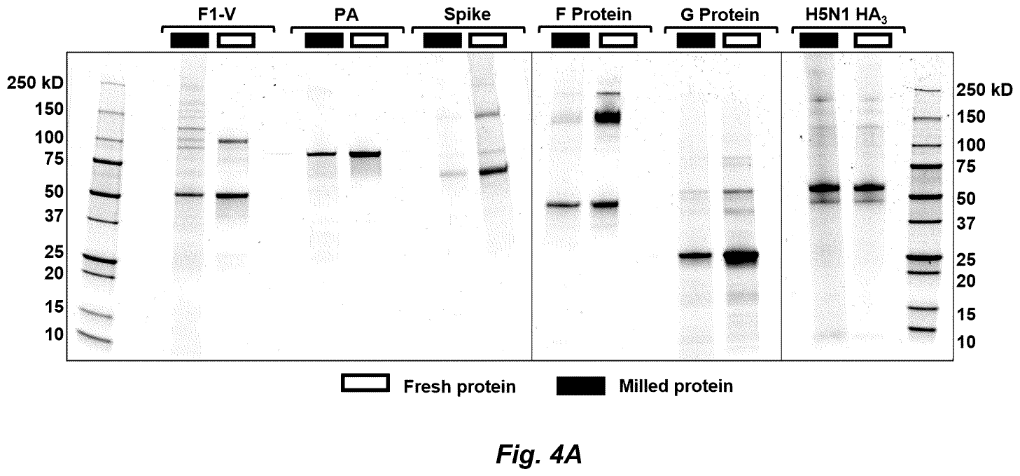

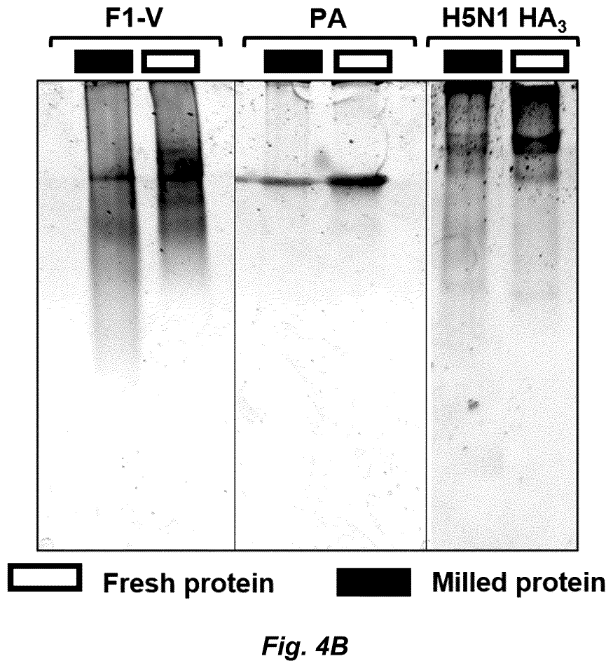

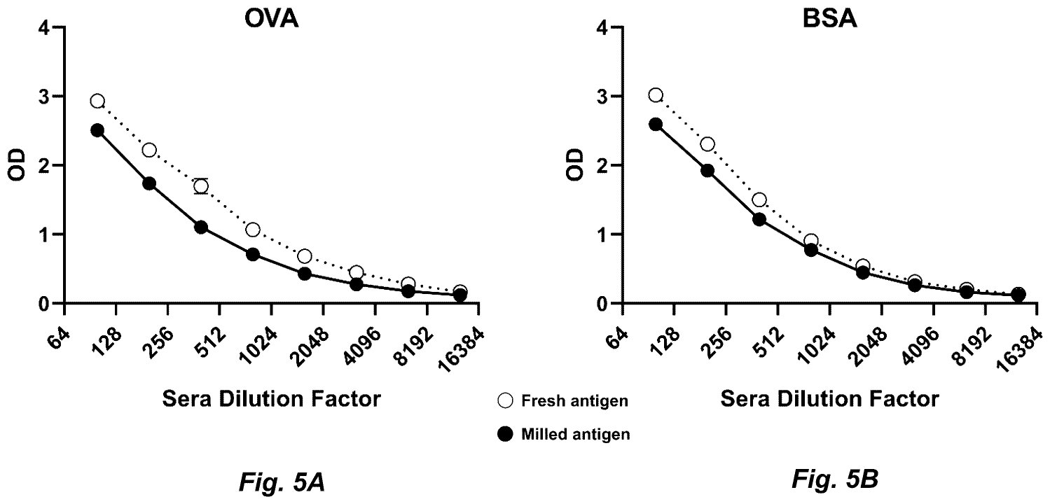

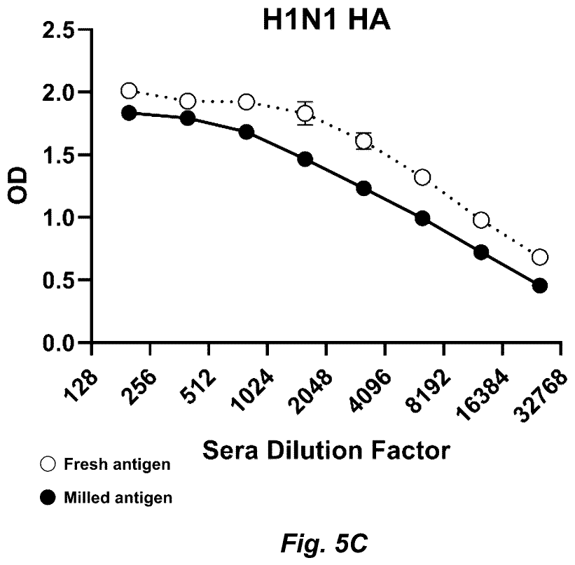

Antigen conformational stability may be critical for the in vivo efficacy of immunogenic PBVs. The stability of protein antigen payloads should therefore be a consideration after undergoing milling to reduce payload aggregates during the PRET process. After milling, a select number of clinically relevant protein antigens were dissolved in an aqueous buffer and SDS-PAGE was performed (FIG. 4A). The primary structure of all proteins observed was maintained at the appropriate molecular weights when compared to fresh, unmilled protein controls. Additionally, native PAGE (FIG. 4B) demonstrates the preservation of native protein structure post-milling, which is especially important for conformational epitopes such as the influenza A virus H5N1 HA trimeric antigen. Finally, an ELISA (FIG. 5) was performed to assess the antigenicity of protein payloads post-milling. In all three examples of OVA, BSA, and influenza A virus H1N1 HA, antigen-specific antibodies were able to recognize and bind to milled antigens similar to fresh, unmilled controls.

Altogether, these results demonstrate the broad applicability of PRET in milling antigenic or non-antigenic payloads to reduce their aggregate size to approximately 1500 nm or less while maintaining stability prior to encapsulation into PBVs.

### Preparation of Payload Via Spray Drying

If milling a payload is undesirable (e.g., payload may be unable to be lyophilized, payload may be incompatible with a known milling solvent, or milling solvent may be immiscible with subsequent polymer solvent), alternative methods such as spray drying may be used to prepare antigenic and non-antigenic payloads to diameters of 1500 nm or less. This can be accomplished by first dissolving the payload in a suitable liquid phase, which is then spray dried using a spray dryer. In general, these types of spray dryers use a piezoelectric mesh nozzle instead of a conical two-phase spray nozzle.

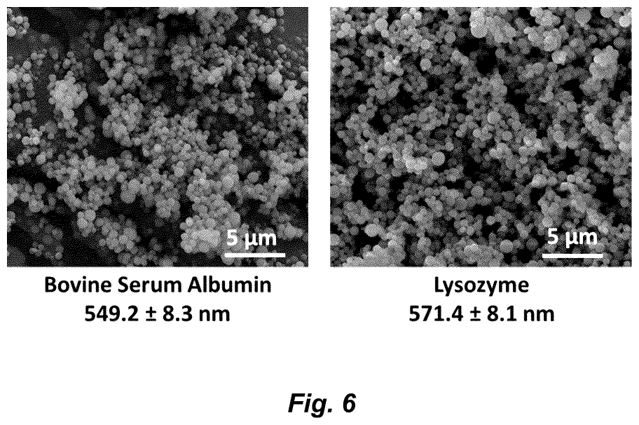

As an example, the model payloads lysozyme and BSA were dissolved in an aqueous solution and spray dried on a nanoparticle spray dryer equipped with a piezoelectric mesh spray nozzle. Image J analysis of SEM micrographs determined the payloads to have an average diameter of 549 nm to 571 nm (FIG. 6), demonstrating a secondary method for preparing a payload size below 1500 nm prior to encapsulation into PBVs.

### Spray Drying PBVs to Encapsulate Prepared Payloads

Once the payloads have been prepared (using wet milling or spray drying) to an average diameter of less than 1500 nm, the payloads are then encapsulated into PBVs via spray drying. In this process, a material, such as a polymer, is dissolved in a suitable solvent. The milled payload suspension or spray dried payload is then added to the polymer solution and mixed to ensure even distribution of the suspended payload throughout the solution. Finally, the solution can be spray dried to form and collect the solid PBV particles encapsulating the payload.

Spray drying conditions for producing PBVs are flexible and should take into consideration the type of polymer, solvent, desired particle size, and batch size before choosing polymer concentration, temperature, feed rate, nozzle size and gas flow, fan speed, or specific spray dryer model. In the following examples, polymers were dissolved in solvent. The milled payload suspension was pipetted into the polymer solution before spray drying.

### PBV Size and Morphology

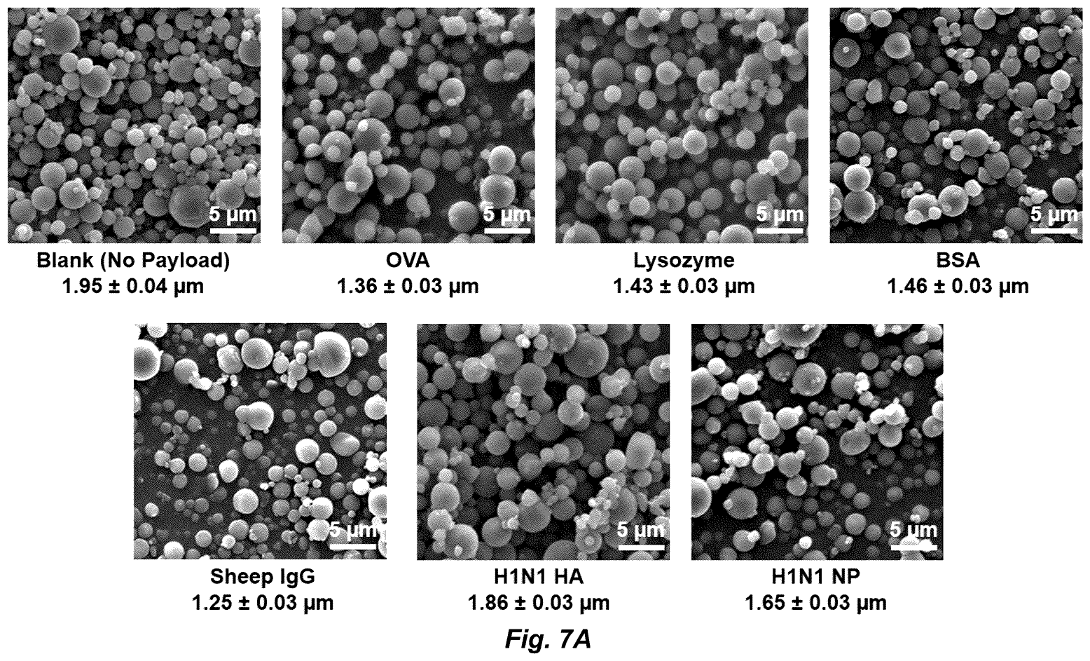

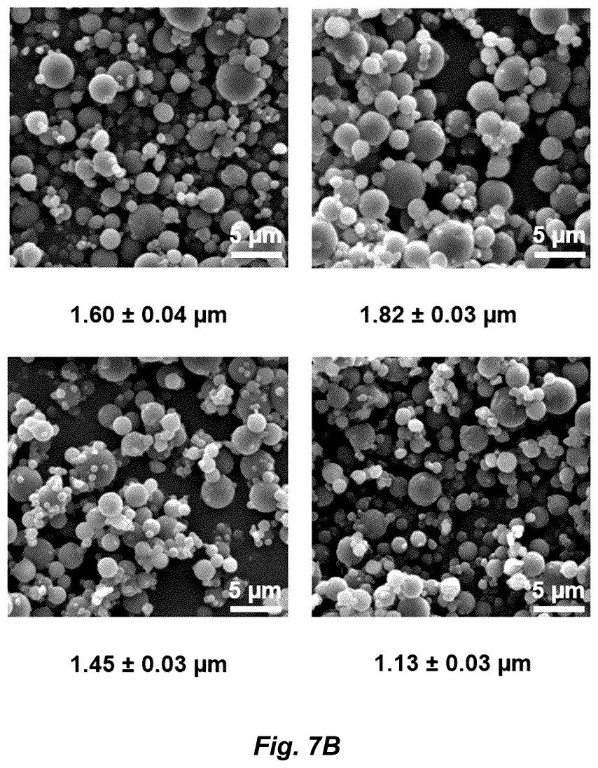

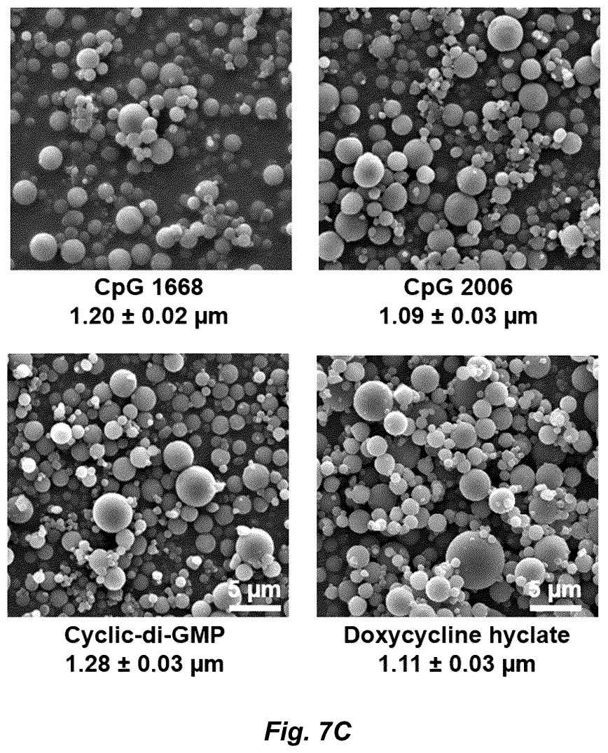

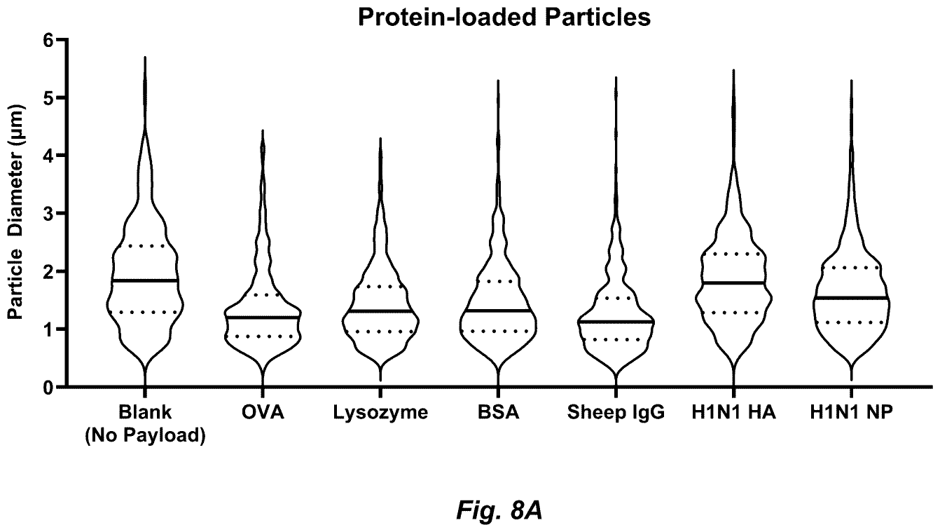

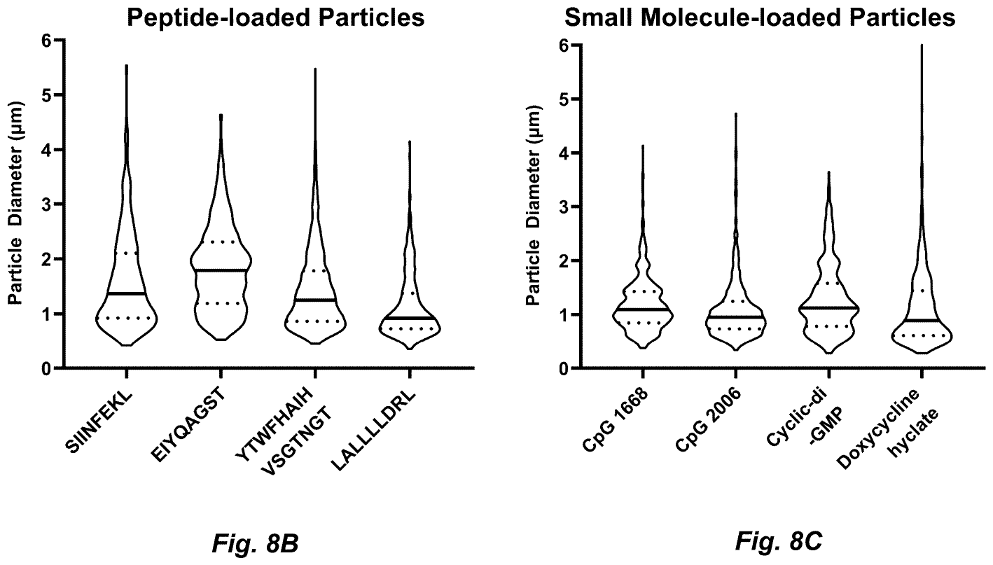

Following using PRET to synthesize 20:80 CPTEG:CPH PBVs encapsulating milled antigenic and non-antigenic payloads, PBV particle size and morphology were characterized via SEM (FIG. 7). Regardless of the payload, all synthesized PBVs were found to have discrete, spherical morphologies. Image J analysis of the SEM micrographs determined all PBV formulations to have an average diameter of 1-2 μm and similar size distributions (FIG. 8).

### Release Kinetics of PRET PBVs

Due to the improved ability of PRET to encapsulate payloads into PBVs, PRET-synthesized particles typically have a minimal burst release and sustained release kinetics of encapsulated payloads. As an example, PBVs encapsulating various payloads were suspended in PBS and the supernatant containing released payload was collected and replaced with fresh buffer over the course of approximately one week. The amount of protein/peptide, CpG oligonucleotide, cyclic-di-GMP, or doxycycline hyclate released at each time point was quantified with microBCA, Qubit fluorometric assay (Thermo Scientific), ELISA (Cayman Chemical, Ann Arbor, MI), or absorbance at 350 nm, respectively. Following 5-8 days of release, the particles were placed in 40 mM sodium hydroxide to rapidly degrade the particles and release the remaining encapsulated payload for quantification.

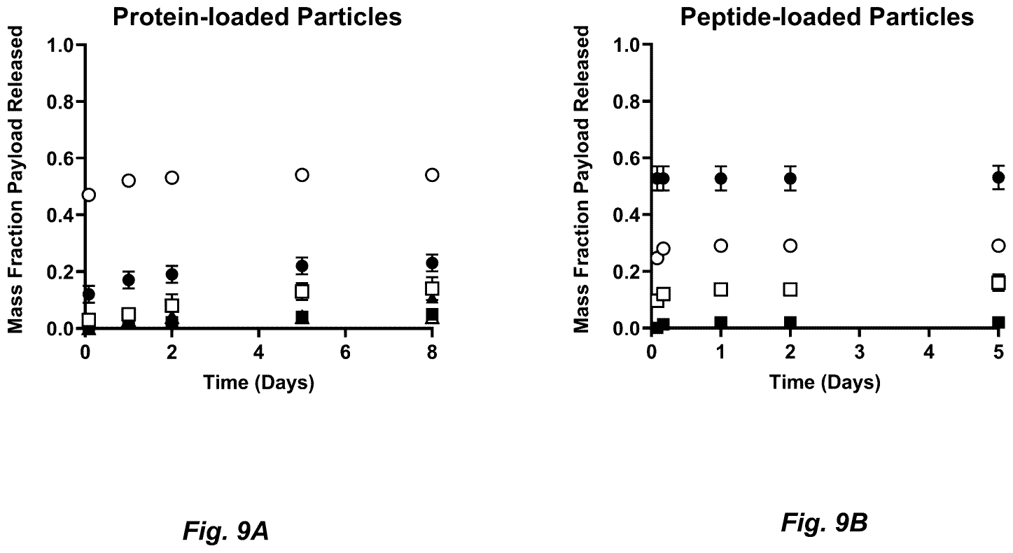

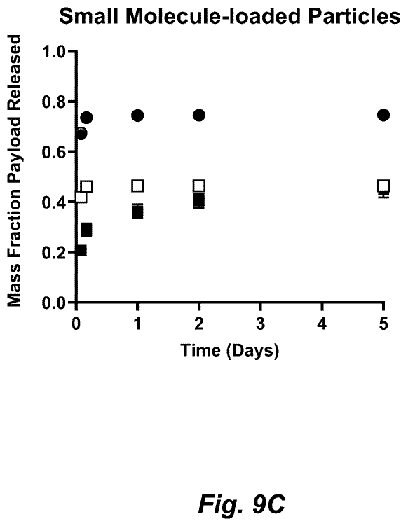

The release kinetics from 20:80 CPTEG:CPH PBVs were found to be payload-dependent (FIG. 9). In general, the PBVs were able to sustain the release of both antigenic and non-antigenic payloads. Interestingly, the encapsulation efficiencies of each encapsulated payloads (Tables 4, 6, and 7) were found to be equivalent or greater than those typically found after traditional, laboratory scale FNP particle synthesis methods for antigenic payloads (30-70%).

TABLE 4
20:80 CPTEG:CPH PBV Encapsulation Efficiencies.

| | Encapsulation |
|---|---|
| Payload | Efficiency |
| OVA | 43.7% |
| BSA | 40.6% |
| Sheep IgG | 44.6% |
| Lysozyme | 37.0% |
| H1N1 HA | 25.5% |
| H1N1 NP | 20.7% |
| SIINFEKL (SEQ ID NO: 1) | 35.0% |
| EIYQAGST (SEQ ID NO: 2) | 10.0% |
| YTWFHAIHVSGTNGT (SEQ ID NO: 3) | 16.2% |
| LALLLLDRL(SEQ ID NO: 4) | 3.6% |
| CpG 1668 (5′-tccatgacgttcctgatg | 64.9% |
| ct-3′) (SEQ ID NO: 5) | |
| CpG 2006 (5′-tcgtcgttttgtcgtttt | 112.9% |
| gtcgtt-3′) (SEQ ID NO: 6) | |
| Cyclic-di-GMP | 48.8% |
| Doxycycline hyclate | 37.6% |

### Stability of PBV-Encapsulated Payloads Upon Release

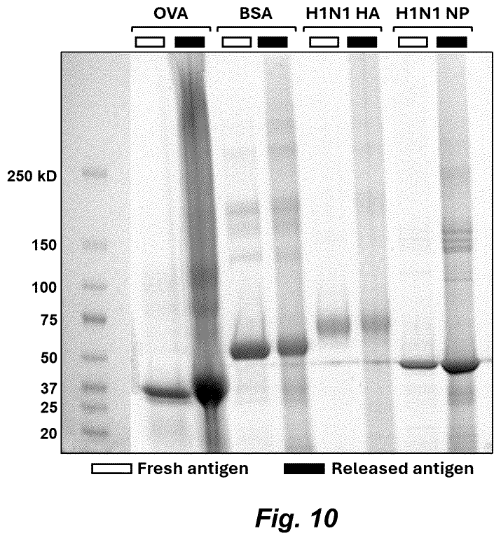

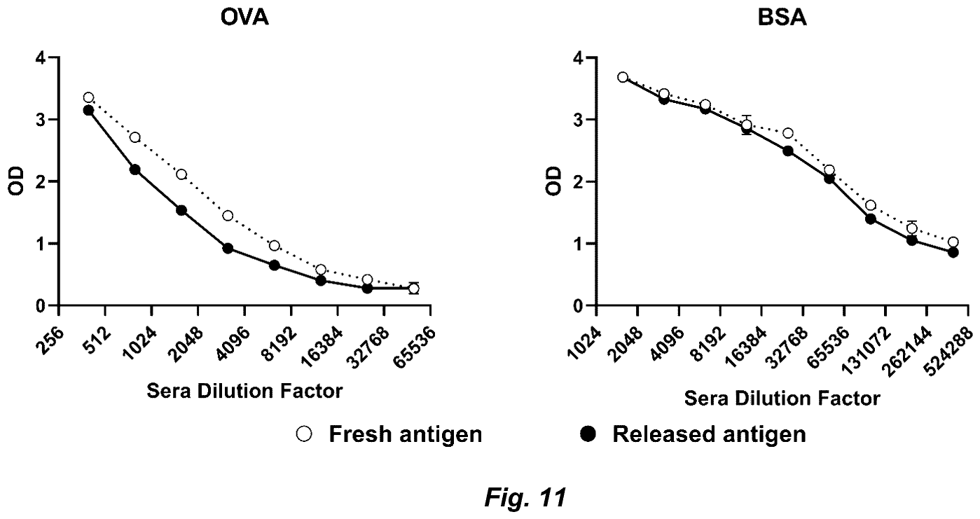

While the PRET process is agnostic to the type of encapsulated payload, it is crucial to ensure payload stability after spray drying, especially for vaccine antigens which may need to remain structurally and conformationally stable to induce effective immune responses. Therefore, SDS-PAGE was performed to assess the primary structure of antigenic payloads OVA, BSA, and influenza A virus H1N1 HA and NP antigens following release from 20:80 CPTEG:CPH PBVs made using PRET. The primary structure of all released proteins was maintained at the appropriate molecular weights when compared to fresh protein controls (FIG. 10). In addition, ELISA analysis of antigens OVA and BSA released from 20:80 CPTEG:CPH PBVs demonstrated that antibodies were able to recognize and bind to these released antigens similar to fresh protein controls (FIG. 11). Therefore, these results demonstrate the ability of PRET in producing PBVs in a reproducible manner that maintains the stability of encapsulated payloads.

### Pret-Synthesized PBVs of Various Polymer Chemistries

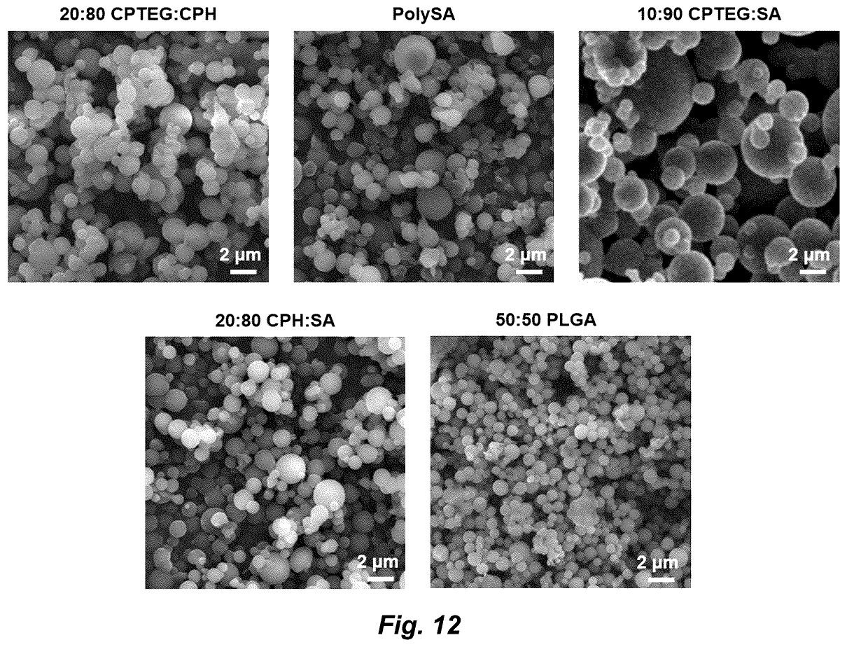

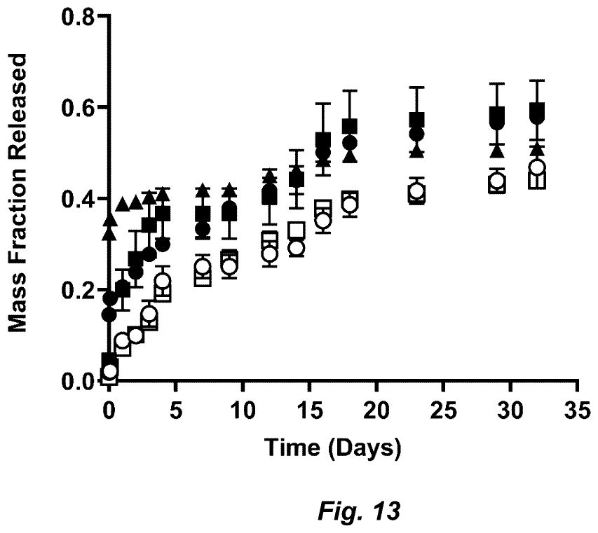

Thus far, this work has focused on demonstrating the broad applicability of the PRET process to various types of encapsulated payloads. Similarly, PRET may encapsulate payloads into a variety of materials, provided that the solvent used in milling is miscible with the solvent used to dissolve the material and maintains the material solubility when combined. To demonstrate this, the model protein OVA was milled and encapsulated into various polymer chemistries using the methods described above. SEM images of the PBVs synthesized using PRET demonstrated consistent size, with diameters of approximately 1-2 μm that did not vary across polymer chemistry (Table 5), including 20:80 CPTEG:CPH, PolySA, 10:90 CPTEG:SA, 20:80 CPH:SA, or 50:50 poly(lactic-co-glycolic) acid (PLGA). In addition, the morphology of the spray dried particles was found to be discrete and spherical, independent of polymer chemistry (FIG. 12). Additionally, the PBVs of the multiple polymer chemistries were incubated in phosphate-buffered saline at 37° C. and supernatants were collected periodically over approximately one month to quantitate released antigen using the microBCA assay. While an initial burst release of antigen varied with polymer chemistry, all PBV formulations were able to sustain antigen release for over 30 days (FIG. 13).

TABLE 5
PBVs of Various Materials.

| PBV Material | Particle Size* (Mean ± St. Dev.) |
|---|---|
| 20:80 CPTEG:CPH | 1.23 ± 0.51 μm |
| PolySA | 1.22 ± 0.54 μm |
| 10:90 CPTEG:SA | 1.81 ± 0.66 μm |
| 20:80 CPH:SA | 1.04 ± 0.39 μm |
| 50:50 PLGA | 0.97 ± 0.22 μm |

*n = 500 particles per sample analyzed

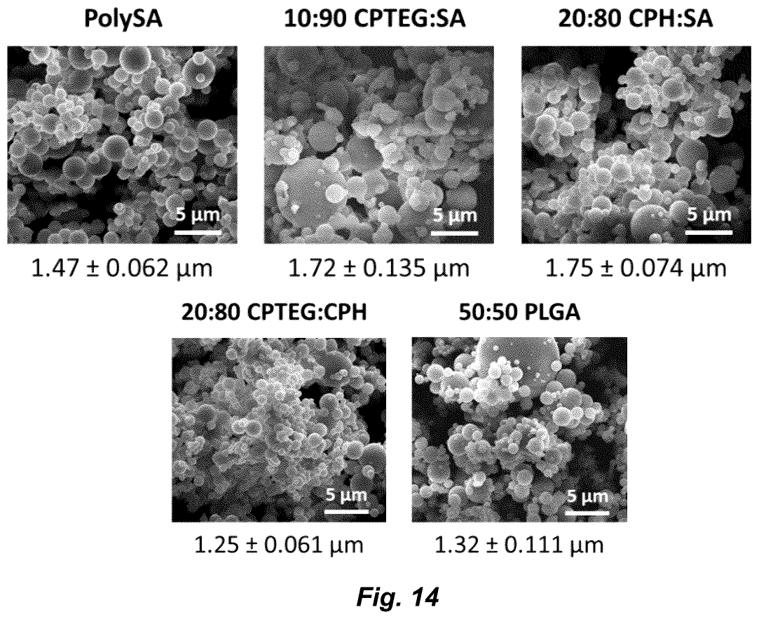

Additionally, payloads that are prepared via spray drying may also be encapsulated into a wide variety of materials. Spray drying payloads may be desirable over wet milling due to an inability of the payload to be either lyophilized without loss of stability, no suitable milling solvent, or inherent immiscibility of the milling and spray dry solvents. To demonstrate this, PBVs were spray dried encapsulating spray dried BSA and lysozyme. Representative images of lysozyme-encapsulated PBVs are shown FIG. 14. As demonstrated with PBVs encapsulating milled payloads, the mean diameters of PBVs encapsulating spray dried lysozyme were between 1-2 μm that did not vary across polymer chemistry. Similarly to the encapsulation efficiency results found using milled payloads, the encapsulation efficiency of spray dried lysozyme was found to be dependent on polymer chemistry; however, all formulations demonstrated high lysozyme encapsulation efficiencies greater than 53% (Table 6).

TABLE 6
Encapsulation Efficiency of Spray Dried Lysozyme.

| PBV Material | Encapsulation Efficiency |
|---|---|
| 20:80 CPTEG:CPH | 71.1% |
| PolySA | 75.8% |
| 10:90 CPTEG:CPH | 71.5% |
| 20:80 CPH:SA | 53.8% |
| 50:50 PLGA | 61.8% |

Co-Encapsulation of Multiple Payloads within PBVs

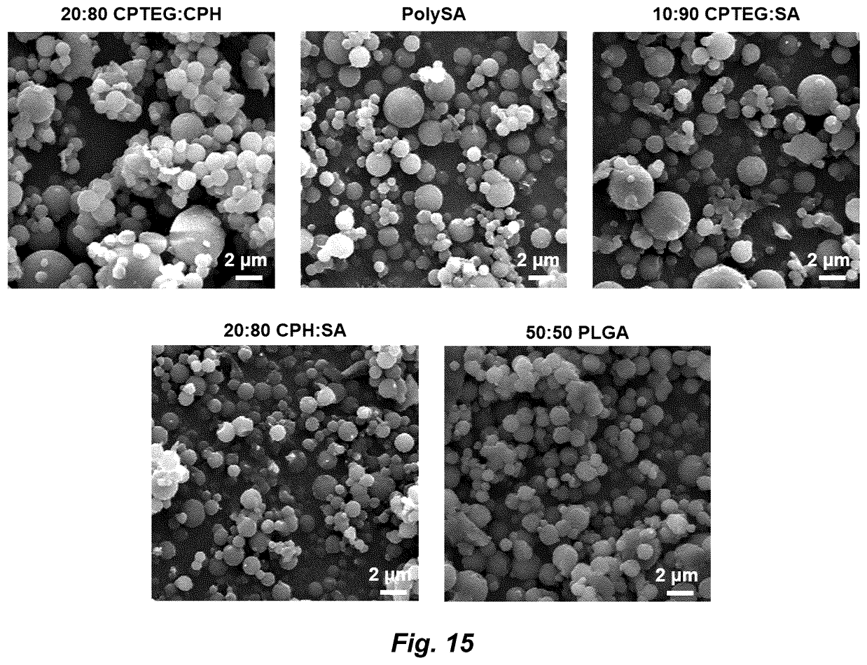

In some cases, it may be necessary to co-encapsulate multiple payloads within the same PBV particle. As an example, co-delivery of a vaccine antigen and adjuvant may be desired to enhance the immune response. As proof of this concept, a model protein antigen (OVA) and co-adjuvant (CpG ODN 1668) were milled following the same conditions described above. The two milled suspensions were added to a single polymer solution before spray drying the PBVs. SEM micrographs of spray dried particles demonstrate that co-encapsulation of multiple payloads did not affect particle size (Table 7) or morphology (FIG. 15). Furthermore, the encapsulation efficiencies of both payloads were determined by placing the PBVs in 40 mM sodium hydroxide and quantifying the release antigen (microBCA) and co-adjuvant (Qubit fluorometric assay). The OVA encapsulation efficiency was found to be dependent on polymer chemistry; however, all formulations demonstrated high OVA encapsulation efficiencies greater than 56%. The encapsulation efficiency of the small molecule co-adjuvant CpG ODN 1668 was also maintained at a high level similar to particles encapsulating CpG ODN 1668 alone, with encapsulation efficiencies greater than 64% (Table 4 vs. 7). It was noted that these encapsulation efficiencies are equivalent or higher than those typically found after traditional laboratory scale FNP particle synthesis methods for antigenic payloads (30-70%).

TABLE 7
PBVs co-encapsulating OVA antigen and CpG ODN 1668 adjuvant

| | Particle Size* | OVA | CpG ODN 1668 |
|---|---|---|---|
| | (Mean ± St. | Encapsulation | Encapsulation |
| PBV Material | Dev.) | Efficiency | Efficiency |
| 20:80 CPTEG:CPH | 1.21 ± 0.58 μm | 58.5% | 105.5% |
| PolySA | 1.04 ± 0.48 μm | 99.9% | 87.2% |
| 10:90 CPTEG:SA | 1.24 ± 0.54 μm | 77.4% | 78.4% |
| 20:80 CPH:SA | 1.09 ± 0.62 μm | 104.5% | 90.6% |
| 50:50 PLGA | 1.02 ± 0.46 μm | 56.7% | 87.4% |

*n = 500 particles per sample analyzed

The examples above demonstrate the ability of PBVs to stabilize encapsulated antigenic payloads at room temperature for extended periods of time (>6 months), reducing the need for refrigeration and the cold chain for vaccine distribution. Moreover, the two-step, high-throughput PRET method for synthesizing PBVs results in a reproducible means of preparing antigenic and non-antigenic payloads via wet milling or spray drying, and their subsequent encapsulation into PBVs. These PBVs demonstrate similar particle size and morphology, irrespective of polymer chemistry. This unique, scalable process is agnostic to payload and particle material, resulting in high encapsulation efficiencies, sustained release kinetics, and stability of encapsulated payloads. Taken together, these two inventions provide a scalable, high-throughput method to synthesize thermostable PBVs with improved consistency and stability during long term, room temperature storage.

All publications, patents, and patent documents cited herein are incorporated by reference as though individually incorporated by reference. No limitations inconsistent with this disclosure are to be understood therefrom. The invention has been described with reference to various specific and preferred embodiments and techniques. However, many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

What is claimed is:

1. A method for manufacturing particle-based compositions comprising the steps of:
i) reducing a lyophilized antigenic and/or a non-antigenic payload to an average size of about 10 nanometers to about 1500 nanometers in the presence of a first solvent to form a payload suspension;
ii) mixing the payload suspension with a solution to form a mixture, wherein the solution comprises a material soluble in a second solvent, wherein the second solvent comprises a solvent suitable for spray drying; and
iii) spray drying the mixture to form the particle-based composition.

2. The method of claim 1, wherein the reducing step comprises milling or grinding the lyophilized antigenic or the non-antigenic payload.

3. The method of claim 2, wherein the reducing step comprises milling, and the milling comprises wet milling.

4. The method of claim 1, wherein the first solvent and the second solvent comprise one or more of an alcohol, a halogenated hydrocarbon, an aliphatic hydrocarbon, an aromatic hydrocarbon, a ketone, an ester, an ether, a nitrile, an amide, and acid, and a base.

5. The method of claim 4, wherein the first solvent and the second solvent comprise one or more of ethanol, chloroform, acetone, dichloromethane, acetonitrile, ethyl acetate, isopropanol, acetic acid, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene.

6. The method of claim 4, wherein the first solvent and the second solvent comprise one or more of ethanol, chloroform, acetone, dichloromethane, acetonitrile, ethyl acetate, and isopropanol.

7. The method of claim 1, wherein a concentration of the antigen or the non-antigenic payload in the milled suspension is about 0.1 mg/mL to about 500 mg/mL.

8. The method of claim 1, wherein a concentration of the material in the mixture is about 0.1 mg/mL to about 500 mg/mL.

9. The method of claim 1, wherein the antigenic payload comprises a protein, a peptide, a nucleic acid, or a small molecule; and/or
the non-antigenic payload comprises one or more of an adjuvant, a protein, peptide, nucleic acid, small molecule, pharmaceutical drug, bacteria, virus, and whole cell lysate.

10. The method of claim 9, wherein the antigenic payload comprises one or more of influenza virus hemagglutinin (HA), influenza virus nucleoprotein (NP), influenza virus neuraminidase (NA), influenza matrix proteins (M), RSV pre- or post-fusion F protein, RSV G protein, or SARS-COV-2 spike protein(S), SARS-COV-2 nucleocapsid (N), SARS-COV-2 membrane protein (M), SARS-COV-2 envelope (E) protein, Yersinia pestis fusion protein F1-V, and Bacillus anthracis protective antigen (PA).

11. The method of claim 9, wherein the non-antigenic payload comprises the adjuvant, wherein the adjuvant is entrapped within the particles, wherein the adjuvant comprises at least one of a CpG oligodeoxynucleotide (CpG ODN), a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, cyclic dinucleotides, and a Stimulator of Interferon Genes (STING) activating adjuvant.

12. The method of claim 1, wherein the material comprises polymers or homopolymers comprising one or more of polyanhydrides, poly(lactic acid) (PLA), poly(glycolic acid) (PGA), poly(lactic-co-glycolic acid) (PLGA), polycaprolactone (PCL), poly(glycerol sebacate) (PGS), PEG-PCL, polyhydroxyalkanoates (PHA), polyethylene (PE), poly(methyl methacrylate) (PMMA), polytetrafluoroethylene (PTFE), silicone, polyurethane, Poly(N-isopropylacrylamide) (PNIPAAm), poly(acrylic acid) (PAA), collagen, chitosan, alginate, hyaluronic acid, chondroitin sulfate, and gelatin.

13. The method of claim 12, wherein the material comprises homopolymers or copolymers comprising an average molecular weight of about 5,000 g/mol to about 30,000 g/mol.

14. The method of claim 1, wherein the material comprises:
i) a CPTEG:CPH copolymer;
ii) a CPTEG:SA copolymer;
iii) a CPH:SA copolymer;
iv) a CPTEG homopolymer;
v) a CPH homopolymer;
vi) a SA homopolymer;
vii) a PLGA copolymer;
viii) a PLA homopolymer;
ix) a PGA homopolymer, or
x) a polymer other than a polyanhydride that is soluble in a solvent suitable for spray drying.

15. A composition manufactured according to the method of claim 1.

16. A composition comprising:
particles comprising one or more materials soluble in a solvent suitable for spray drying; and
one or more antigenic and/or non-antigenic payloads entrapped within the particles; wherein the one or more antigenic and/or the non-antigenic payloads have an average size of about 10 nm to about 1500 nm;
wherein the particles have an average particle size of about 0.15 μm to about 10 μm.

17. The composition of claim 17, wherein the one or more materials comprise one or more of a biodegradable material, a nonbiodegradable material, a chemically degradable material, a stimuli-responsive material, a water-soluble material, and a swellable material.

18. The composition of claim 17, wherein the material comprises one or more of polyanhydrides, poly(lactic acid) (PLA), poly(glycolic acid) (PGA), poly(lactic-co-glycolic acid) (PLGA), polycaprolactone (PCL), poly(glycerol sebacate) (PGS), PEG-PCL, polyhydroxyalkanoates (PHA), polyethylene (PE), poly(methyl methacrylate) (PMMA), polytetrafluoroethylene (PTFE), silicone, polyurethane, Poly(N-isopropylacrylamide) (PNIPAAm), poly(acrylic acid) (PAA), collagen, chitosan, alginate, hyaluronic acid, chondroitin sulfate, and gelatin.

19. The composition of claim 16, wherein the material comprises homopolymers or copolymers comprising an average molecular weight of about 5,000 g/mol to about 30,000 g/mol.

20. The composition of claim 16, wherein the solvent suitable for spray drying comprises one or more of chloroform, dichloromethane, ethanol, acetone, acetonitrile, ethyl acetate, isopropanol, acetic acid, acetonitrile, benzene, 2-butanone, n-butanol, butyl acetate, carbon tetrachloride, cyclohexane, 1,2-dichloroethane, diethyl ether, di-isopropyl ether, dimethylformamide, dimethyl sulfoxide, dioxane, heptane, hexane, isooctane, methanol, methyl ethyl ketone, methyl-t-butyl ether, pentane, n-propanol, tetrahydrofuran, toluene, trichloroethylene, water, and xylene.

21. The composition of claim 20, wherein the solvent suitable for spray drying comprises one or more of chloroform, dichloromethane, ethanol, acetone, acetonitrile, ethyl acetate, and isopropanol.

22. The composition of claim 16, wherein the antigenic payload comprises a eukaryotic, a prokaryotic, or viral antigen.

23. The composition of claim 22, wherein the antigenic payload comprises one or more of influenza virus hemagglutinin (HA), influenza virus nucleoprotein (NP), influenza virus neuraminidase (NA), influenza matrix proteins (M), RSV pre- or post-fusion F protein, RSV G protein, or SARS-COV-2 spike protein(S), SARS-COV-2 nucleocapsid (N), SARS-COV-2 membrane protein (M), SARS-COV-2 envelope (E) protein, Yersinia pestis fusion protein F1-V, and Bacillus anthracis protective antigen (PA).

24. The composition of claim 17, wherein the non-antigenic payload comprises the adjuvant, wherein the adjuvant is entrapped within the particles, wherein the adjuvant is a CpG oligodeoxynucleotide (CpG ODN), a Toll-like receptor (TLR) agonist, a nucleotide oligomerization domain (NOD)-like receptor (NLR) agonist, a RIG-like receptor (RLR), a liposome, an aluminum salt, a mineral salt, an oil emulsion, a polymer, a polysaccharide, a saponin, a cyclic dinucleotide, or a Stimulator of Interferon Genes (STING) activating adjuvant.

25. The composition of claim 18, wherein the material comprises polyanhydrides, and the particles are stable at room temperature for at least 6 months, wherein the polyanhydrides are selected from the group consisting of:
i) a CPTEG:CPH copolymer;
ii) a CPTEG:SA copolymer;
iii) a CPH:SA copolymer;
iv) a CPTEG homopolymer;
v) a CPH homopolymer; and
vi) a SA homopolymer.
